SEQUENCE LISTING (99 sequences)

SEQ ID NO: 1 - protein, 631 aa, Alicyclobacillus acidocaldarius [sequence omitted]

SEQ ID NO: 2 - protein, 631 aa, Thermesynechococcus elongatus
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQVSISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAFDVDNTSDLPNHIPFCIFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTTFPGDFYAGYTMYRHVFPTLALGRYKQAIERR

SEQ ID NO: 3 - protein, 658 aa, Zymomonas mobilis
MTVSTSSAFHHSPLSDDVEPIIQKATRALLEKQHQDGHWVFELEADATIPAEYILLKHYLGEPQDLEIEAKIGRYLRRIQGEHGGWSLFYGGDLDLSATVKAYFALKMIGDSPDAPHMLRARNEILARGGAMRANVFTRIQLALFGAMSWEHVPQMPVELMLMPEWFPVHINKMAYWARTVLVPLLVLQALKPVARNRRGILVDELFVPDVLPTLQESGDPIWRRFFSALDKVLHKVEPYWPKNMRAKAIHSCVHFVTERLNGEDGLGAIYPAIANSVMMYDALGYPENHPERAIARRAVEKLMVLDGTEDQGDKEVYCQPCLSPIWDTALVAHAMLEVGGDEAEKSAISALSWLKPQQILDVKGDWAWRRPDLRPGGWAFQYRNDYYPDVDDTAVVTMAMDRAAKLSDLRDDFEESKARAMEWTIGMQSDNGGWGAFDANNSYTYLNNIPFADHGALLDPPTVDVSARCVSMMAQAGISITDPKMKAAVDYLLKEQEEDGSWFGRWGVNYIYGTWSALCALNVAALPHDHLAIQKAVAWLKTIQNEDGGWGENCDSYALDYSGYEPMDSTASQTAWALLGLMAVGEANSEAVTKGINWLAQNQDEEGLWKEDYYSGGGFPRVFYLRYHGYSKYFPLWALARYRNLKKANQPIVHYGM

SEQ ID NO: 4 - protein, 631 aa, Alicyclobacillus acidocaldarius
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTTFPGDFYAGYTMYRHVFPTLALGRYKQAIERR

SEQ ID NO: 5 - protein, 631 aa, Alicyclobacillus acidocaldarius
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAFDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTTFPGDFYAGYTMYRHVFPTLALGRYKQAIERR

SEQ ID NO: 6 - protein, 631 aa, Alicyclobacillus acidocaldarius
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQVSISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAFDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTTFPGDFYAGYTMYRHVFPTLALGRYKQAIERR

SEQ ID NO: 7 - protein, 631 aa, Alicyclobacillus acidocaldarius; identical to SEQ ID NO: 2

SEQ ID NO: 8 - protein, 631 aa, Alicyclobacillus acidocaldarius
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTTFPGDFYLGYTMYRHVFPTLALGRYKQAIERR

SEQ ID NO: 9 - protein, 631 aa, Alicyclobacillus acidocaldarius
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTAFPGDFYLGYTMYRHVFPTLALGRYKQAIERR

SEQ ID NO: 10 - protein, 631 aa, Alicyclobacillus acidocaldarius
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTSFPGDFYLGYTMYRHVFPTLALGRYKQAIERR

SEQ ID NO: 11 - protein, 631 aa, Alicyclobacillus acidocaldarius
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTNFPGDFYLGYTMYRHVFPTLALGRYKQAIERR

SEQ ID NO: 12 - protein, 631 aa, Alicyclobacillus acidocaldarius
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTDFPGDFYLGYTMYRHVFPTLALGRYKQAIERR

SEQ ID NO: 13 - protein, 631 aa, Alicyclobacillus acidocaldarius
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTQFPGDFYLGYTMYRHVFPTLALGRYKQAIERR

SEQ ID NO: 14 - protein, 631 aa, Alicyclobacillus acidocaldarius
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTEFPGDFYLGYTMYRHVFPTLALGRYKQAIERR

SEQ ID NO: 15 - protein, 631 aa, Alicyclobacillus acidocaldarius
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTCFPGDFYLGYTMYRHVFPTLALGRYKQAIERR

SEQ ID NO: 16 - protein, 631 aa, Alicyclobacillus acidocaldarius
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTRFPGDFYLGYTMYRHVFPTLALGRYKQAIERR

SEQ ID NO: 17 - protein, 631 aa, Alicyclobacillus acidocaldarius
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTNFPGDFYSGYTMYRHVFPTLALGRYKQAIERR

SEQ ID NO: 18 - protein, 631 aa, Alicyclobacillus acidocaldarius
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTMFPGDFYLGYTMYRHVFPTLALGRYKQAIERR

SEQ ID NO: 19 - protein, 631 aa, Alicyclobacillus acidocaldarius
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTLFPGDFYLGYTMYRHVFPTLALGRYKQAIERR

SEQ ID NO: 20 - protein, 631 aa, Alicyclobacillus acidocaldarius
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTYFPGDFYLGYTMYRHVFPTLALGRYKQAIERR

SEQ ID NO: 21 - protein, 631 aa, Alicyclobacillus acidocaldarius
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTKFPGDFYLGYTMYRHVFPTLALGRYKQAIERR

SEQ ID NO: 22 - protein, 631 aa, Alicyclobacillus acidocaldarius
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTVFPGDFYLGYTMYRHVFPTLALGRYKQAIERR

SEQ ID NO: 23 - protein, 631 aa, Alicyclobacillus acidocaldarius
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTFFPGDFYLGYTMYRHVFPTLALGRYKQAIERR

SEQ ID NO: 24 - protein, 631 aa, Alicyclobacillus acidocaldarius
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTIFPGDFYLGYTMYRHVFPTLALGRYKQAIERR

SEQ ID NO: 25 - protein, 631 aa, Alicyclobacillus acidocaldarius
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTWFPGDFYLGYTMYRHVFPTLALGRYKQAIERR

SEQ ID NO: 26 - protein, 631 aa, Alicyclobacillus acidocaldarius; identical to SEQ ID NO: 9

SEQ ID NO: 27 - protein, 631 aa, Alicyclobacillus acidocaldarius
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTPFPGDFYLGYTMYRHVFPTLALGRYKQAIERR

SEQ ID NO: 28 - protein, 631 aa, Alicyclobacillus acidocaldarius
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTGFPGDFYSGYTMYRHVFPTLALGRYKQAIERR

SEQ ID NO: 29 - protein, 631 aa, Alicyclobacillus acidocaldarius
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTGFPGDFYMGYTMYRHVFPTLALGRYKQAIERR

SEQ ID NO: 30 - protein, 631 aa, Alicyclobacillus acidocaldarius
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTGFPGDFYAGYTMYRHVFPTLALGRYKQAIERR

SEQ ID NO: 31 - protein, 631 aa, Alicyclobacillus acidocaldarius
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTGFPGDFYVGYTMYRHVFPTLALGRYKQAIERR

SEQ ID NO: 32 - protein, 631 aa, Alicyclobacillus acidocaldarius; identical to SEQ ID NO: 30

SEQ ID NO: 33 - protein, 631 aa, Alicyclobacillus acidocaldarius
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAFDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTGFPGDFYAGYTMYRHVFPTLALGRYKQAIERR

SEQ ID NO: 34 - protein, 631 aa, Alicyclobacillus acidocaldarius
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAFDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTTFPGDFYAGFTMYRHVFPTLALGRYKQAIERR

SEQ ID NO: 35 - protein, 631 aa, Alicyclobacillus acidocaldarius
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTGFPGDFYLGFTMYRHVFPTLALGRYKQAIERR

SEQ ID NO: 36 - DNA, 31 bp, Alicyclobacillus acidocaldarius
ccgtattataccggcacctgcttcccgggcg

SEQ ID NO: 37 - DNA, 31 bp, Alicyclobacillus acidocaldarius
cgcccgggaagcaggtgccggtataatacgg

SEQ ID NO: 38 - DNA, 31 bp, Alicyclobacillus acidocaldarius
ccgtattataccggcaccgatttcccgggcg

SEQ ID NO: 39 - DNA, 31 bp, Alicyclobacillus acidocaldarius
cgcccgggaaatcggtgccggtataatacgg

SEQ ID NO: 40 - DNA, 31 bp, Alicyclobacillus acidocaldarius
ccgtattataccggcaccgaattcccgggcg

SEQ ID NO: 41 - DNA, 31 bp, Alicyclobacillus acidocaldarius
cgcccgggaattcggtgccggtataatacgg

SEQ ID NO: 42 - DNA, 31 bp, Alicyclobacillus acidocaldarius
ccgtattataccggcacccatttcccgggcg

SEQ ID NO: 43 - DNA, 31 bp, Alicyclobacillus acidocaldarius
cgcccgggaaatgggtgccggtataatacgg

SEQ ID NO: 44 - DNA, 31 bp, Alicyclobacillus acidocaldarius
ccgtattataccggcaccattttcccgggcg

SEQ ID NO: 45 - DNA, 31 bp, Alicyclobacillus acidocaldarius
cgcccgggaaaatggtgccggtataatacgg

SEQ ID NO: 46 - DNA, 31 bp, Alicyclobacillus acidocaldarius
ccgtattataccggcaccaaattcccgggcg

SEQ ID NO: 47 - DNA, 31 bp, Alicyclobacillus acidocaldarius
cgcccgggaatttggtgccggtataatacgg

SEQ ID NO: 48 - DNA, 31 bp, Alicyclobacillus acidocaldarius
ccgtattataccggcaccatgttcccgggcg

SEQ ID NO: 49 - DNA, 31 bp, Alicyclobacillus acidocaldarius
cgcccgggaacatggtgccggtataatacgg

SEQ ID NO: 50 - DNA, 31 bp, Alicyclobacillus acidocaldarius
ccgtattataccggcaccaacttcccgggcg

SEQ ID NO: 51 - DNA, 31 bp, Alicyclobacillus acidocaldarius
cgcccgggaagttggtgccggtataatacgg

SEQ ID NO: 52 - DNA, 31 bp, Alicyclobacillus acidocaldarius
ccgtattataccggcaccccgttcccgggcg

SEQ ID NO: 53 - DNA, 31 bp, Alicyclobacillus acidocaldarius
cgcccgggaacggggtgccggtataatacgg

SEQ ID NO: 54 - DNA, 31 bp, Alicyclobacillus acidocaldarius
ccgtattataccggcacccagttcccgggcg

SEQ ID NO: 55 - DNA, 31 bp, Alicyclobacillus acidocaldarius
cgcccgggaactgggtgccggtataatacgg

SEQ ID NO: 56 - DNA, 31 bp, Alicyclobacillus acidocaldarius
ccgtattataccggcaccctattcccgggcg

SEQ ID NO: 57 - DNA, 31 bp, Alicyclobacillus acidocaldarius
cgcccgggaatagggtgccggtataatacgg

SEQ ID NO: 58 - DNA, 31 bp, Alicyclobacillus acidocaldarius
ccgtattataccggcacctcgttcccgggcg

SEQ ID NO: 59 - DNA, 31 bp, Alicyclobacillus acidocaldarius
cgcccgggaacgaggtgccggtataatacgg

SEQ ID NO: 60 - DNA, 31 bp, Alicyclobacillus acidocaldarius
ccgtattataccggcaccaccttcccgggcg

SEQ ID NO: 61 - DNA, 31 bp, Alicyclobacillus acidocaldarius
cgcccgggaaggtggtgccggtataatacgg

SEQ ID NO: 62 - DNA, 31 bp, Alicyclobacillus acidocaldarius
ccgtattataccggcaccgtgttcccgggcg

SEQ ID NO: 63 - DNA, 31 bp, Alicyclobacillus acidocaldarius
cgcccgggaacacggtgccggtataatacgg

SEQ ID NO: 64 - DNA, 31 bp, Alicyclobacillus acidocaldarius
ccgtattataccggcacctatttcccgggcg

SEQ ID NO: 65 - DNA, 31 bp, Alicyclobacillus acidocaldarius
cgcccgggaaataggtgccggtataatacgg

SEQ ID NO: 66 - DNA, 39 bp, Alicyclobacillus acidocaldarius
ggcgatttttatgcgggctttaccatgtatcgccatgtg

SEQ ID NO: 67 - DNA, 38 bp, Alicyclobacillus acidocaldarius
cacatggcgatacatggtaaagcccgcataaaaatcgc

SEQ ID NO: 68 - DNA, 40 bp, Alicyclobacillus acidocaldarius
caacggcggctggggcgcgtttgatgtggataacaccagc

SEQ ID NO: 69 - DNA, 40 bp, Alicyclobacillus acidocaldarius
gctggtgttatccacatcaaacgcgccccagccgccgttg

SEQ ID NO: 70 - DNA, 42 bp, Alicyclobacillus acidocaldarius
ggttcccgggcgatttttatgccatgtataccatgtatcgcc

SEQ ID NO: 71 - DNA, 42 bp, Alicyclobacillus acidocaldarius
ggcgatacatggtatagccggcataaaaatcgcccgggaacc

SEQ ID NO: 72 - DNA, 42 bp, Alicyclobacillus acidocaldarius
ggttcccgggcgatttttatagccattataccatgtatcgcc

SEQ ID NO: 73 - DNA, 42 bp, Alicyclobacillus acidocaldarius
ggcgatacatggtatagccgctataaaaatcgcccgggaacc

SEQ ID NO: 74 - DNA, 42 bp, Alicyclobacillus acidocaldarius
ggttcccgggcgatttttattatggctataccatgtatcgcc

SEQ ID NO: 75 - DNA, 42 bp, Alicyclobacillus acidocaldarius
ggcgatacatggtatagccataataaaaatcgcccgggaacc

SEQ ID NO: 76 - DNA, 42 bp, Alicyclobacillus acidocaldarius
ggttcccgggcgatttttgtcccatgtataccatgtatcgcc

SEQ ID NO: 77 - DNA, 42 bp, Alicyclobacillus acidocaldarius
ggcgatacatggtatagccgggacaaaaatcgcccgggaacc

SEQ ID NO: 78 - DNA, 41 bp, Alicyclobacillus acidocaldarius
  misc_feature (22)..(22): n is a, c, g, or t
ggttcccgggcgatttttatrntggctataccatgtatcgc

SEQ ID NO: 79 - DNA, 39 bp, Alicyclobacillus acidocaldarius
ataaaaatcgcccgggaacccggtgccggtataatacgg

SEQ ID NO: 80 - DNA, 43 bp, Alicyclobacillus acidocaldarius
  misc_feature (21)..(21): n is a, c, g, or t
gcgatggcagctggggcggcndtcagccgccgtggttttatgc

SEQ ID NO: 81 - DNA, 43 bp, Alicyclobacillus acidocaldarius
gcgatggcagctggggcggcvhgcagccgccgtggttttatgc

SEQ ID NO: 82 - DNA, 43 bp, Alicyclobacillus acidocaldarius
gcgatggcagctggggcggctggcagccgccgtggttttatgc

SEQ ID NO: 83 - DNA, 37 bp, Alicyclobacillus acidocaldarius
tgccgccccagctgccatcgcccgcctggcgttccag

SEQ ID NO: 84 - DNA, 47 bp, Alicyclobacillus acidocaldarius
  misc_feature (20)..(20): n is a, c, g, or t
gaaaccgggcggctttgcgndtcagtttgataacgtgtattatccgg

SEQ ID NO: 85 - DNA, 47 bp, Alicyclobacillus acidocaldarius
gaaaccgggcggctttgcgvhgcagtttgataacgtgtattatccgg

SEQ ID NO: 86 - DNA, 47 bp, Alicyclobacillus acidocaldarius
gaaaccgggcggctttgcgtggcagtttgataacgtgtattatccgg

SEQ ID NO: 87 - DNA, 36 bp, Alicyclobacillus acidocaldarius
cgcaaagccgcccggtttcaggttcgggcgtttcac

SEQ ID NO: 88 - DNA, 39 bp, Alicyclobacillus acidocaldarius
  misc_feature (19)..(19): n is a, c, g, or t
ggctattggtggggcccgndtctgagcaacgtgaccatg

SEQ ID NO: 89 - DNA, 39 bp, Alicyclobacillus acidocaldarius
ggctattggtggggcccgvhgctgagcaacgtgaccatg

SEQ ID NO: 90 - DNA, 39 bp, Alicyclobacillus acidocaldarius
ggctattggtggggcccgtggctgagcaacgtgaccatg

SEQ ID NO: 91 - DNA, 39 bp, Alicyclobacillus acidocaldarius
cgggccccaccaatagccttcatctttctggcagctcag

SEQ ID NO: 92 - DNA, 37 bp, Alicyclobacillus acidocaldarius
  misc_feature (19)..(19): n is a, c, g, or t
ggctggatgtttcaggcgndtattagcccggtgtggg

SEQ ID NO: 93 - DNA, 37 bp, Alicyclobacillus acidocaldarius
ggctggatgtttcaggcgvhgattagcccggtgtggg

SEQ ID NO: 94 - DNA, 37 bp, Alicyclobacillus acidocaldarius
ggctggatgtttcaggcgtggattagcccggtgtggg

SEQ ID NO: 95 - DNA, 37 bp, Alicyclobacillus acidocaldarius
cgcctgaaacatccagccgccataatccagttccacg

SEQ ID NO: 96 - DNA, 36 bp, Alicyclobacillus acidocaldarius
  misc_feature (19)..(19): n is a, c, g, or t
ggcggctggatgtttcagndtagcattagcccggtg

SEQ ID NO: 97 - DNA, 36 bp, Alicyclobacillus acidocaldarius
ggcggctggatgtttcagvhgagcattagcccggtg

SEQ ID NO: 98 - DNA, 36 bp, Alicyclobacillus acidocaldarius
ggcggctggatgtttcagtggagcattagcccggtg

SEQ ID NO: 99 - DNA, 42 bp, Alicyclobacillus acidocaldarius
ctgaaacatccagccgccataatccagttccacgccatacag

(19) United States
(12) Patent Application Publication
(10) Pub. No.: US 2024/0018505 A1
(43) Pub. Date: Jan. 18, 2024

(54) ENZYMATIC MONOCYCLIZATION OF ACYCLIC MONOTERPENOIDS

(71) Applicant: Univesität Stuttgart, Stuttgart (DE)

(72) Inventors: Bernhard Hauer, Fußönheim (DE); Andreas Schneider, Stuttgart (DE)

(21) Appl. No.: 18/037,136

(22) PCT Filed: Nov. 18, 2021
(86) PCT No.: PCT/EP2021/082104

§ 371 Date: May 16, 2023

(30) Foreign Application Priority Data
Nov. 18, 2020 (EP) 20208314.3
Mar. 1, 2021 (EP) 21160100.0

(51) Int. Cl. C12N 9/90; C12P 5/00

(52) U.S. Cl. CPC C12N 9/90; C12Y 504/99017; C12P 5/007

(57) ABSTRACT

Enzyme mutant with squalene-hopene-cyclase activity, selected from mutants of a wild-type enzyme comprising an amino acid sequence selected from SEQ-ID No: 1 to 3 or an amino acid sequence derived therefrom with a degree of sequence identity in the range of from 60 to 99.9% of SEQ-ID No. 1 to 3, wherein the mutant catalyzes a one-step monocyclization reaction to produce products such as gamma-dihydroionone and/or alpha-dihydroionone.

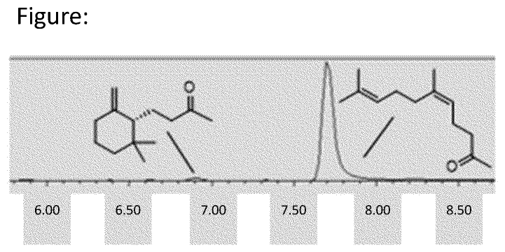

The present invention relates to novel enzyme mutants with cyclase activity and to a process for the monocyclization of acyclic monoterpenoids using the novel enzyme mutants.

Further embodiments of the present invention relate to nucleic acid sequences encoding for the novel enzyme mutants, to expression cassettes comprising such nucleic acid sequences and to a recombinant vector comprising, under the control of a regulative element, at least one nucleic acid sequence in accordance with the present invention or at least one expression cassette in accordance with the present invention.

Furthermore, the present invention relates to a recombinant microorganism comprising at least one nucleic acid sequence in accordance with the present invention or at least one expression cassette in accordance with the present invention or at least one recombinant vector in accordance with the present invention.

The cyclization of terpenes with specific cyclases is known per se. As an example, squalene is cyclized to the pentacyclic compound hopene with a squalene-hopene cyclase in a process occurring in nature.

Apocarotenoids are naturally occurring isoprenoid structures derived from oxidative cleavage of several C40-carotenoids. The shorter analogues of those, mainly consisting of less than 15 carbon atoms are often used in the flavor and fragrances industry for personal care products or food flavouring agents and are therefore of high economic interest.

Rose ketones like Damascones 5-7, Damascenones 8 or violet-type odor molecules like Ionones 1-3 and Dihydroionones 4 have an outstanding position in this field due to their extremely low odor threshold.

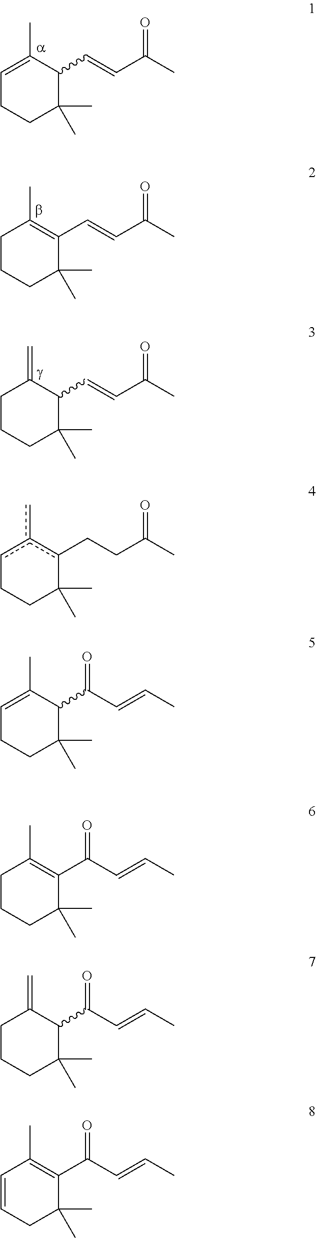

This property together with the odor itself is closely related to the overall structure of the molecule and therefore an easy accessibility by chemical synthesis is highly desirable. The ionones 1-3 as the longest known and most studied apocarotenoids are either extracted as a mixture of isomers from plants, derived from oxidative cleavage of higher molecular carotenoids or synthesized by acid-catalyzed cyclization of their linear precursor. Hereby the choice of the acid determines the resulting product α-1, β-2 or γ-ionone 3 using phosphoric, sulfuric or a lewis acid e.g. trifluoro boric acid. All of them differ in their olfactory properties whereby the (S)-γ-derivative has the most powerful and pleasant odor. In order to obtain the (S)-γ-ionone 3 a stereoselective cyclization would be preferable, but is still a challenge in classical organic chemistry and as a result multistep synthesis and lipase-catalyzed resolution approaches are commonly used.

The corresponding dihydroionones (4) are comparable in their properties to those of the ionones (1-3). Subsequently the most prominent derivative here is also the γ-derivative 9. It is a photooxidative degradation product of ambrein, which is a main component of ambergris, also known as “floating gold” due to their high value on the market.

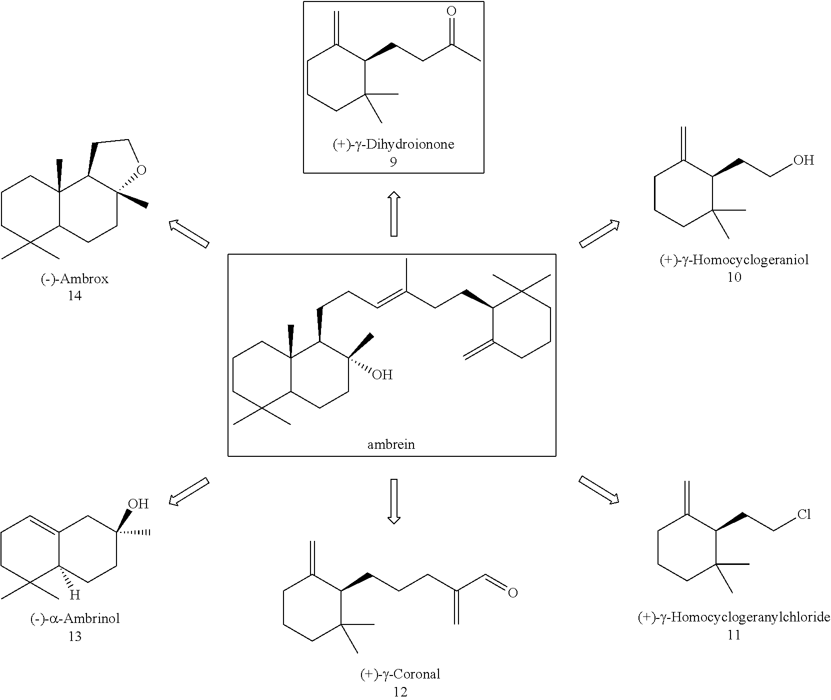

Compound (+)-9 can act as a precursor to chemically synthesize 10, 11, 12 in three steps and enriched 13 in a one-step cyclization. In order to obtain (+)-9 the precursor ionone must be chemically reduced with hydrogen in presence of Raney-Nickel or otherwise irradiated with a high-pressure Hg-lamps. Direct cyclization approaches from commercially available geranyl acetone (16t) suffer from inevitably required epoxy-derivatives, protection groups or end up in bicyclic or multicyclic products. As a result (+)-9 is basically unavailable on the market due to cost-intensive and tedious synthesis.

In general, two approaches for the generation of apocarotenoids have been described in the literature: On the one hand there is the chemical approach which is divided into heterogeneous and homogeneous catalysis based on acidic cyclization and on the other hand there are some biotechnological approaches based on carotenoid cleaving enzymes.

Chemical Approaches

The earliest preparation method for the Dihydroionones (4) known in literature is the reduction of the corresponding precursor Ionone. Francke et al. described the hydrogenation of (+)-1 to the reduced compound (+)-α-Dihydroionone with a poor ee of 18% (Francke, W., Schulz, S., Sinnwell, V., König, W. A., & Roisin, Y, Epoxytetrahydroedulan, a new terpenoid from the hairpencils of Euploea (Lep.: Danainae) butterflies. Liebigs Annalen der Chemie 1989.12, 1195-1201 (1989)). In 2000 Fuganti et al. (Fuganti, C., Serra, S. & Zenoni, A. Synthesis and olfactory evaluation of (+)- and (−)-γ-ionone. Helv. Chim. Acta 83, 2761-2768 (2000)) first evaluated the olfactory properties of (+/−)-γ-Ionone 3 and (+/−)-γ-Dihydroionone 9 by synthesizing racemic 3 over four steps, perform crystallization and lipase-catalyzed resolution and finally reduce the enantiomerically pure ionone via Palladium catalysis to get enantiopure (+)-9 with 6% overall yield.

Tsangarakis et al. (Tsangarakis, C. & Stratakis, M. Biomimetic cyclization of small terpenoids promoted by zeolite NaY: Tandem formation of α-ambrinol from geranyl acetone. Adv. Synth. Catal. 347, 1280-1284 (2005)) investigated the NaY promoted cyclization of the small terpenoids Geraniol, Geranyl acetate, Farnesyl acetate and Geranyl acetone 16t. The catalyst was completely unselective and no yields for α-Dihydroionone are given as this compound was not present in more than insignificant amounts in the reaction mixture.

In 2008 Justicia (Justicia, J. et al. Titanium-catalyzed enantioselective synthesis of α-ambrinol. Adv. Synth. Catal. 350, 571-576 (2008)) and co-workers presented the first enantioselective synthesis of (−)-α-Ambrinol 13 starting from Geranyl acetone 16t. The overall sequence resulted in 18% yield over 8 chemical steps.

Another method for synthesis of regioisomerically enriched γ-Dihydroionone 9 was described by Serra, S., Fuganti, C. & Brenna, E. Two easy photochemical methods for the conversion of commercial ionone alpha into regioisomerically enriched γ-ionone and γ-dihydroionone. Flavour Fragr. J. 22, 505-511 (2007). The authors used commercial α-Ionone 1, reduced and acetylated this substrate and finally irradiated the dihydro-α-ionol acetate to get enriched 9. The method needs 6-9 steps and lacks enantioselectivity.

In Serra, S. “An expedient preparation of enantio-enriched ambergris odorants starting from commercial ionone alpha”, Flavour Fragr. J. 28, 46-52 (2013). racemic α-Ionone 1 again was used as a starting agent and was transformed in either 4 steps to enriched (+)-γ-Ionone 3 or 5 steps to enriched (+)-9. The overall yield of the synthesis sequence was about 16%.

Biotechnological Approaches

The only biotechnological synthesis of Dihydroionones 4 was reported by Sanchez-Contreras, A., Jiménez, M. & Sanchez, S. Bioconversion of lutein to products with aroma. Appl. Microbiol. Biotechnol. 54, 528-534 (2000). A colony from marigold flower dehydration mud which was capable of degrading lutein was isolated. Two microorganisms were assigned to catalyze the reaction: Trichosporon asahii for the carotenoid cleavage of lutein and Paenibacillus amylolyticus for the reduction of the resulting cleavage products.¹⁸ The product mixture contained besides β-Ionone 2, 3-hydroxy-β-Ionone and Dihydro-β-Ionol also traces of Dihydro-β-ionone with around 3% relative conversion related to lutein.

The squalene-hopene cyclases (SHCs) belong to the family of triterpene cyclases, which catalyze the polycyclization of linear terpenes and terpenoids. Together with the oxidosqualene cyclases (OSCs) and the diterpene cyclases, it belongs to the protonase superfamily. They initiate the reaction by protonating a prenyl group, which makes them class II terpene cyclases. The natural substrate of the bacterial SHC from Alicyclobacillus acidocaldarius is the triterpene squalene, which after protonation undergoes a cascade-like, concerted polycyclization to give pentacyclic hopene or hopanol. The complexity of this one-step reaction in the formation of nine stereocenters and twelve new bonds in the pentacyclic framework of hopene or hopanol in a 5:1 ratio. This reaction is promoted, among other things, by the pre-folding of linear squalene into an all-pre-chair conformation in the active site. On the other hand, the intermediary carbocation is stabilized by aromatic amino acids. The substrate scope of this enzyme ranges from elongated C35 terpenes to the small terpenoid geraniol. Besides isoprenyl protonation, also carbonyls and epoxides can be activated and other reaction types like isomerizations, Prins reactions and Friedel-Crafts alkylations can be catalyzed.

There still exists a need to efficient and economically feasible synthesis routes to monocyclize apocarotenoids to obtain e.g. γ-Dihydroionone 9 and other monocyclic compounds which can be used as flavors and fragrances.

This object is achieved with enzyme mutants in accordance with claim 1.

Preferred embodiments of the invention are set forth in the dependent claims and the detailed specification hereinafter.

In the first embodiment, the present invention relates to enzyme mutants with Squalene-Hopene cyclase activity, selected from mutants of a wild-type enzyme comprising an amino acid sequence selected from SEQ-ID No. 1 to 3 or a partial sequence thereof or an amino acid sequence derived from SEQ-ID No. 2 to 3 with a degree of sequence identity in the range of from 60 to 99.9%, preferably in the range of from 70 to 99.9% to SEQ-ID No. 2 to 3, wherein the mutant catalyzes at least the one-step monocyclization of a substrate of general formula (I)

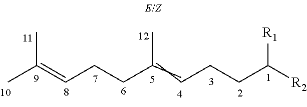

to a monocyclic compound of formula (II)

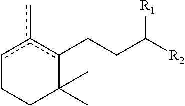

wherein at least one of substituents R¹ und R² is selected from the group consisting of oxo, —OH, thiol, amino, ester, halogen, nitro or nitrile groups and wherein at least one of substituents R¹ und R² is selected from hydrogen, alkyl or alkylene groups.

Alkyl groups preferably comprise from 1 to 10, more preferably from 1 to 8 and most preferably from 1 to 6 carbon atoms. Representative examples are methyl, ethyl, propyl, 1-methylethyl, butyl, 1-mathylpropyl, 2-methylpropyl, 1,1-dimethylethyl, pentyl, 1-methylbutyl, 2-methylbutyl, 3-methylbutyl, 2,2-dimethylpropyl, 1-ethylproypyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl.

Alkenyl groups preferably comprise from 2 to 20, more preferably from 2 to 10 and even more preferably from 2 to 8 carbon atoms and represent linear or branched hydrocarbon residues with one or more double bonds. Representative examples are ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 3-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2 propenyl.

The term oxo defines a substituent forming a keto group together with the carbon atom to which it is bound.

The enzyme mutant in accordance with the present invention has a squalene-hopene-cyclase activity, i.e. it catalyzes the cyclization of squalene to hopene. Squalene-hopene-cyclases (also referred to as SHC hereinafter) have been described in the literature and are classified as EC. 5.4.99.17 in the EC enzyme classification scheme.

The term cyclase-activity as used herein refers to an enzyme activity determined under standard conditions with a reference substrate which describes the formation of a cyclic product starting from a non-cyclic product. Standard conditions are substrate concentration, pH value and temperature. The measurement can be made with recombinant cyclase-expressing cells, fractions thereof or with the purified enzyme.

The compounds of formula (I) which are used as substrate in accordance with the present invention belong to the group of acyclic monoterpenoids.

These compounds are monoterpenes that do not contain a cycle.

The present invention encompasses, but is not limited to enzyme mutants with squalene-hopene-cyclase activity catalyzing the monocyclization of substrates of formula (I) and comprising an amino acid sequence SEQ-ID No. 1 to 3 or a partial sequence thereof comprising e.g. at least 50, 100, 150, 200, 250, 300, 350, 400, 450, 500, 550 or 600 consecutive amino acid residues of one of these sequences.

The degree of homology to sequence ID Nos 1 to 3 is at least 60, more preferably at least 75 and even more preferably at least 90, 91, 92, 93, 94, 95, 96, 97, 98, 99%. Homology is determined using the algorithm of Pearson and Litman, Proc. Natl. Acad. Sci USA 85(8), 1988, 2444-2448. A homology respectively identity of an enzyme mutant in % in accordance with the present invention in particular means a respective identity of the amino acid groups based on the entire length of the amino acid sequence.

In accordance with a first preferred embodiment, up to 40%, preferably up to 30%, more preferably up to 20% of the amino acid groups in the enzyme mutant are modified compared to SEQ-ID No. 1 or SEQ-ID No. 2 to 3 by deletion, insertion, substitution, addition or a combination thereof.

In accordance with another preferred embodiment of the invention, the enzyme mutant comprises
a) a mutation in position G600 of SEQ-ID No. 1 or
b) a mutation in an amino acid sequence selected from amino acid sequences SEQ-ID Nos 2 to 3 wherein the position (referred to hereinafter also as equivalent position) of the mutation corresponds to position G600 of SEQ-ID No. 1.

The term functional mutants, as used herein, relates to enzyme mutants comprising at least one mutation in an equivalent position as defined above.

Preferred mutations in position G600 of SEQ-ID. No. 1 or in a position corresponding to position G600 in SEQ-ID. No. 1 in one of the amino acid sequences SEQ-ID. No. 2 to SEQ-ID 3 is a substitution selected from the group consisting of G600A (SEQ-ID No. 9), G600S (SEQ-ID No.10), G6000 (SEQ-ID No.15), G600T (SEQ-ID No. 8), G600N (SEQ-ID No.11), G600D (SEQ-ID No.12), G600Q (SEQ-ID No.13) und G600E (SEQ-ID No.14).

The enzyme mutants in accordance with the present invention may, in addition to the mutation in position G600 of SEQ-ID No. 1 or a corresponding to position G600 in SEQ-ID. No. 1 in one of the amino acid sequences SEQ-ID. No. 2 to SEQ-ID 3 further comprise at least one mutation in one of positions Y420 or L607 of SEQ-ID. No. 1, for example enzyme mutants of SEQ-ID Nos 4 (G600T/L607A) and 5 (G600T/L607A/Y420F), or at least one mutation in an amino acid sequence selected from amino acid sequences SEQ-ID Nos 2 to 3 wherein the position of the mutation corresponds to position Y420 respectively L607 of SEQ-ID No. 1.

Further preferred enzyme mutants in accordance with the present invention comprise three mutations in positions Y420, G600 und L607 of Sequence-ID. No. 1, for example enzyme mutant of SEQ-ID No. 5, or three mutations in an amino acid sequence selected from amino acid sequences SEQ-ID Nos 2 to 3 wherein the position of the mutation corresponds to position Y420, G600 and L607 of SEQ-ID No. 1 or comprise four mutations in positions A306, Y420, G600 und L607 of Sequence-ID. No. 1, for example enzyme mutant of SEQ-ID No. 6 (G600T/L607A/Y420F/A306V), or four mutations in an amino acid sequence selected from amino acid sequences SEQ-ID Nos 2 to 3 wherein the position of the mutations correspond to positions A306, Y420, G600 and L607 of SEQ-ID No. 1 or five mutations in positions A306, Y420, D436, G600 and L607 of Sequence-ID. No. 1, for example enzyme mutant of SEQ-ID No. 7 (G600T/L607A/Y420F/A306V/D4361), or five mutations in an amino acid sequence selected from amino acid sequences SEQ-ID Nos 2 to 3 wherein the position of the mutations correspond to positions A306, Y420, D436, G600 and L607 of SEQ-ID No. 1.

Particularly preferred enzyme mutants comprise an amino acid sequence selected from SEQ-ID. Nos. 4 to 8.

Other aspects of the present invention relate to a nucleic acid sequence, encoding for an enzyme mutant in accordance with the present invention, to expression cassettes, comprising a nucleic acid sequence encoding for an enzyme mutant in accordance with the present invention, to a recombinant vector, comprising under the control of at least one regulative element, at least one nucleic acid sequence encoding for an enzyme mutant in accordance with the present invention or comprising at least one expression cassette, comprising a nucleic acid sequence encoding for an enzyme mutant in accordance with the present invention.

Furthermore, the present invention relates to a recombinant microorganism comprising at least one nucleic acid sequence in accordance with the present invention or at least one expression cassette in accordance with the present invention or at least one recombinant vector in accordance with the present invention.

Sequence ID No. 1 represents the sequence of the squalene hopene cyclase from Alicyclobacillus acidocaldarius (hereinafter AacSHC), which catalyzes the polycyclization of linear C₃₀ terpene squalene to pentacyclic hopene/hopanol building up nine stereocenters enantiopure.

In the course of the present invention, it was found that position G600 is a hot spot position for smaller substrate conversion and the Arginine having bulky but fairly flexible properties.

Surprisingly we found that mainly small polar amino acids drive the monocyclization reaction of acyclic monoterpenoids of formula (I), e.g. geranyl acetone 16. We assume this is facilitated due to hydrogen-bonding of the Threonine residue to the carbonyl-group of the substrate or the corresponding position in other substrates.

While the invention is described hereinafter by reference to geranyl acetone as one possible substrate of formula (I), the enzyme mutants in accordance with the present invention are generally capable to convert acyclic monoterpenoids of general formula (I) to monocyclic products of formula (II).

The substrates of formula (I) include all isomeric forms of the respective compounds, i.e. constitutional isomers, stereoisomers or their mixtures such as optical isomers or geometrical isomers such as E- and Z-isomers as well as any combinations thereof. If the substrate comprises more than one asymmetric center all combinations of different conformations of such asymmetric centers are possible, such as e.g. pairs of enantiomers.

The E- and Z-isomer of geranyl acetone (16c and 16t) were converted with variant G600R to bicyclic products as major products and, to a smaller extent, to the monocyclic products α-(17)- and γ-dihydroionone (9)

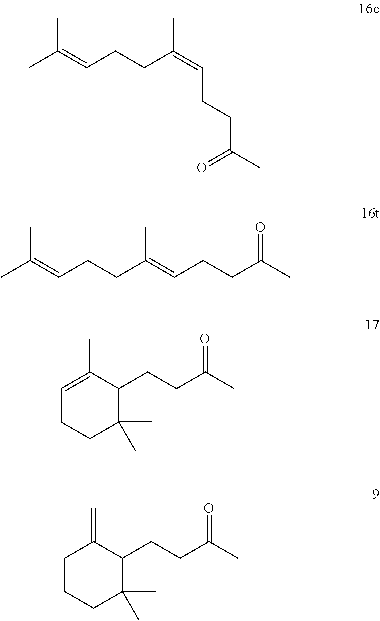

In the course of the present invention, it has been found that mainly small and polar amino acids drive the monocyclization reaction at position G600 and the variant G600T performed best in terms of selectivity. In particular the variants G600T (SEQ-ID No.8), G600N (SEQ-ID No.11), G6000 (SEQ-ID No.15), G600S (SEQ-ID No.10), G600D (SEQ-ID No.12), G600E (SEQ-ID No. 14) and in small amounts the unpolar G600A (SEQ-ID No. 9) generated monocylic γ-dihydroionone 9 and/or α-dihydroionone 17.

Docking studies of the substrate 16c in the active site of the variant G600T (SEQ-ID No. 8) with YASARA were carried out and suggest two major pre-folding states: Pre-folding state 1 favors bicyclization due to the coordination of the carbonyl moiety by the Y420-hydroxy group. The resulting second carbocation of the cation cascade reaction may interact with one lone-pair of the oxygen and thereby form a covalent bond. Bulky amino acids at position G600 favor this pre-folding state. However, there is a turning point at the size of threonine, where polarity seems to play a more significant role. It is currently assumed that this is due to hydrogen bonding capabilities of polar residues. Pre-folding state 2 shows the carbonyl moiety hydrogen-bonded by G600T and Y609 thus the lone-pairs of the oxygen are faced away from the resulting second carbocation ultimately resulting in monocyclic products. Furthermore, steric interaction of the C1-methyl group of the substrate 16c and the Leucine at position 607 can be assumed in this pre-folding state.

Based on the docking results site-directed mutagenesis at the position L607 was introduced and the results revealed that smaller amino acids than leucine in this position are beneficial for the monocyclization reaction.

Site-directed mutagenesis of variant G600T (SEQ-ID No. 8) with the degenerated codon RVT (encoding only smaller acids than leucine, e.g. Alanine, Asparagine, Aspartate, Glycine, Isoleucine, Serine, Threonine, Valine at position L607) revealed variant G600T/L607A (SEQ-ID No. 4, hereinafter N1) showing almost double total turnover number to monocyclic products compared to G600T (SEQ-ID No. 8) and higher selectivity (79%) towards the desired γ-dihydroionone 9 (G600T: 50%).

Interestingly the substrates containing a hydroxy-moiety instead of a carbonyl (geranyl isopropanol and calmusol) show better conversion towards monocyclic compounds with the variant G600N/L607A. This could be due to the ability of the Asparagine at position 600 to also accept hydrogen-bonds instead of only donating them as in the case of Threonine. The monocyclization of geranyl isopropanol turned out be better with variant G600T (SEQ-ID No. 8) than G600T/L607A (SEQ-ID No.4.) As this substrate lacks the keto-methyl group it should not be dependent on increased space at position L607.

Introducing a third mutation in position Y420 yielding variant G600T/L607A/Y420F (SEQ-ID No. 5, hereinafter N2) doubled the direct turnover to monocyclic products compared to variant G600R/L607T and a selectivity of 94% towards the desired product γ-dihydroionone 9. Bicyclization was reduced to only 2% and the traces of the hydration product were completely eliminated.

Introducing a fourth mutation in position A306 yielded variant G600T/L607A/Y420F/A306V (SEQ-ID No. 6, hereinafter referred to as neryl acetone monocyclase or NMC) which showed a nearly 160-fold increased total turnover number compared to the wild-type and 97% selectivity towards the desired product γ-dihydroionone 9.

Sulfuric acid-catalyzed cyclization experiments of γ-dihydroionone 12 to gain (+/−)-α-ambrinol 13 depending on the starting material revealed the enantiopure product (+)-α-ambrinol 13 and therefore enantiopure (−)-γ-dihydroionone 9. Thus, the NMC-catalyzed reaction does not only proceed with high total turnover and product selectivity, but also enantioselectivity is tightly controlled with >99.5% ee.

Hydrogen-bond-knock-out-variants with the variant N2 (as the product selectivity was already very high at this point) via site-directed mutagenesis at positions 600 and 609 were prepared. The variant K0600 (T600 knocked out) showed almost 80% less conversion and 22% less selectivity towards the desired product 9. Variant K0609 (Y609 knocked out) lost almost full activity and 50% of selectivity towards the monocyclization. Single-point mutation variant Y609F (T600 and Y609 knocked out, Y420 switched on) showed no conversion at all.

The mutational experiments and the computational can be explained by the following mechanism:

After the substrate 16c enters the active site its carbonyl moiety is loosely coordinated by the Threonine at position 600. By this attractive interaction, the carbonyl moiety flips into the direction of the Tyrosine at position 609 for tight binding by a strong hydrogen bond. This hydrogen-bond mediated conformation allows the highly product- and enantioselective monocyclization of neryl acetone 16c in one catalytic step. The increased space at position 607 (Leucine→Alanine) creates some space for the C1-methyl group of the substrate on the one hand and can possibly shorten the distance between T600 and Y609 on the other hand. Both scenarios solely or in combination favor the carbonyl moiety flip to the Tyrosine. The mediating role of the T600 is emphasized by the variant K0600 which still shows some conversion of the substrate with good selectivity. Together with the disabled hydrogen-bond donor at position 420 (Tyrosine→Phenylalanine) this would explain the residual good selectivity towards the desired monocyclic product 9. The key role of the strong hydrogen binding Y609 is underlined by the variant K0609 and Y609F. The variant K0609 almost lost its full activity, due to weak coordination of the carbonyl moiety by the Threonine at position 600 and the latter enzyme showed no activity towards monocyclic product at all.

The potential of the engineered NMC and some variants (G600T, G600N/L607S, N1, N2) generated on the engineering pathway was investigated. E/Z-mixtures of substrate analoga were used for the biotransformations. Neryl acetone 16c was best converted by NMC in terms of monocyclization. The substrates containing a hydroxy-moiety instead of a carbonyl showed better conversion towards monocyclic compounds with the variant G600N/L607S (SEQ-ID No.17). This is presumably due to the fact that the asparagine's and hydroxyl-moiety binding capabilities match better than that of threonine and a hydroxyl-moiety. The monocyclization of substrate 18 (formula (I) with R¹ being ═O and R² being hydrogen towards compound 19 turned out be better with variant G600T (SEQ-ID No. 8) than G600T/L607A (SEQ-ID No. 4). Product 19 lacks, compared to 16, the keto-methyl group and thus should not be dependent on less steric bulk at position L607.

The scalability of this reaction could be demonstrated by converting 2 g (2.24 ml) of neryl acetone 16c with the engineered variant NMC with still high selectivity (95%; 1% α-product 14) towards the desired product 12. Isolation of the product (89% yield) and subsequent cyclization confirmed enantiopure conversion. Interestingly when using the E/Z-mixture of geranyl acetone 16 the evolved NMC converts the Z-isomer 16c prior to the E-isomer 16t. This is to our knowledge the first Z-selective type II cyclase reported in the literature.

The results of the experiments show the easy scalability, of the monocyclization of substrates of formula (I) to the desired monocyclic products by using the enzyme mutants in accordance with the present invention which provides an interesting perspective for industrial purposes to obtain products of formula (II) which are currently not easily accessible.

The results furthermore show the applicability of inducing pre-folding by attraction of the substrate's polar functional group. At the same time they resemble the importance of choice of the polar functional group for creating hydrogen bonds as not every hydrogen bond has the same power.

The present invention thus provides a catalytic one-step product- and enantioselective abortive cyclization of compounds of formula (I) towards compounds of formula (II) in gram-scale. This reaction is enabled by engineering polar functional groups inside the active site of AacSHC, thus adding the ability to anchor polar functional groups of non-natural substrates via hydrogen-bonding. This novel feature of protonases allows the enzyme to induce non-natural pre-folding and result in abortive cyclizations products. These findings set the fundament for the evolution of protonases to control and abort complex non-natural cationic cyclization cascades for desired terpene products.

A further subject of the present invention are nucleic acid sequences encoding for an enzyme mutant in accordance with the present invention as defined in the claims and described in detail hereinbefore.

These nucleic acid sequences (e.g. single or double stranded DNA and RNA-sequences such as cDNA and mRNA) may be obtained as described in the literature such as e.g. by fragmental condensation of single overlapping complementary nucleic acid constituents of the double helix or by any other method described in the literature for the manufacture or isolation of nucleic acids. The chemical synthesis of oligonucleotides is known to the skilled person so that no further details need to be given here.

The invention also includes nucleic acid fragments which can be used as hybridizing probes or primers for the identification or amplification of nucleic acid sequences in accordance with the present invention.

The present invention furthermore relates to expression cassettes comprising a nucleic acid sequence in accordance with the present invention. An expression cassette is an expression unit which is functionally linked to the nucleic acid or the gene to be expressed. Thus, an expression cassette encompasses not only nucleic acid sequences regulating transcription and translation but also nucleic acid sequences which, as a result of translation and transcription are intended to be expressed as a protein.

Preferably, such expression cassettes comprise a promotor in 5-direction relative to the encoding sequence and a terminator sequence in 3-direction relative to the encoding sequence, and, eventually further regulative elements functionally linked to the encoding sequence.

An expression cassette in accordance with the present invention can e.g. be obtained by fusion of a suitable promotor with a suitable nucleotide sequence with a terminator signal. Respective recombination and cloning techniques are described e.g. in T. Maniatis, E. F. Fritsch, and J. Sambrook, “Molecular Cloning: A laboratory manual, Cold Spring Harbor Laboratory, Cold Spring Harbor (N.Y.), 1989.

The present invention furthermore relates to recombinant vectors, comprising under the control of at least one regulative element, at least one nucleic acid sequence in accordance with the present invention or at least one expression cassette in accordance with the present invention. The term vector, as used in the present invention, comprises plasmids and phages as well as any other vectors known to the skilled person such as viruses such as CV40, baculovirus and adenovirus, transposons, IS elements, phasmids, cosmids and linear or circular DNA and which may be replicated autonomously in the host organism or by chromosomes.

Suitable plasmids are e.g. described in WO 2012/066059 on page 37.

Another aspect of the present invention are recombinant microorganisms comprising at least one nucleic acid sequence in accordance with the present invention or at least one expression cassette in accordance with the present invention or at least one recombinant vector in accordance the present invention. The term microorganism encompasses wild-type microorganisms as well as genetically modified recombinant microorganisms.

As recombinant host organisms for the nucleic acid sequence or the expression cassette in accordance with the present invention any prokaryotic or eucaryotic organisms are principally suitable. Preferably bacteria, fungi or yeasts are used. Particularly preferred are gram-positive or gram-negative bacteria, more preferably bacteria of the group consisting of the families enterobacteriaceae, pseudomonaceae, rhizobiaceae, streptomycetaceae or nocardiaceae, in particular of the groups escherichia, pseudomonas, Streptomyces, nocardia, burkholderia, salmonella, agrobacterium, clostridium or rhodococcus with Escherichia coli being particularly preferred.

Another subject of the present invention is a process for the manufacture of compounds of formula (II)

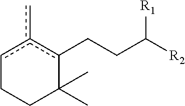

wherein at least one of substituents R¹ and R² is selected from the group consisting of oxo, —OH, thiol, amino, ester, halogen, nitro or nitrile groups and wherein at least one of substituents R¹ und R² is selected from hydrogen, alkyl or alkylene groups wherein compounds of formula (I)

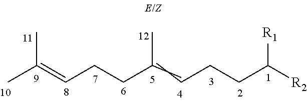

wherein R¹ and R² are as defined above, are cyclized with an enzyme mutant in accordance with the present invention or in the presence of a microorganism expressing an enzyme mutant in accordance with the present invention.

The skilled person will select the best suitable reaction conditions for the process in accordance with the present invention based on his professional knowledge so that in principle no further details need to be given here. Exemplary process conditions may be taken form the working examples, which constitute embodiments of the present invention.

The present invention provides enzyme mutants which are particularly suitable for the monocyclization of acyclic monoterpenoids such as e.g. geranyl acetone 16 to compounds of formula (II) in good yield and high purity, in particular high isomeric or enantiomeric purity. The compounds of formula (II) are of particular interest in the flavor and fragrance industry.

The present invention thus for the first time provides a process for the manufacture of the compounds of formula (II) starting from natural materials by a biotechnological process which is much simpler and faster than the conventional chemical routes known in the art. Complex multistep processes with low yield are replaced by simple one-step processes with good yield and purity of the desired products.

Further preferred embodiments are the subject-matter of the dependent claims. The invention is in some more detail described in the following examples and the accompanying FIGURE.

The FIGURE shows the conversion of substrate 16 with squalene-hopene cyclase from Thermesynechococcus elongatus (TeSHC).

### WORKING EXAMPLES

Material

Chemicals:

The chemicals used for syntheses, molecular biology and biochemical work have been purchased from Carl-Roth (Karlsruhe, DE), VWR (Pennsylvania, US), Sigma-Aldrich (St. Louis, US) and Alfa-Aesar (Ward Hill, US). The substrates (E/Z)-geranyl acetone from VWR (A19184.14), Calmusal from ambinter (18445-88-0) and ambrinol from Amyris. All the other subtrates for biocatalytic purposes were chemically synthesized and analyzed by ¹H-NMR, ¹³C-NMR and GC/MS.

Molecular Biological Kits.

The molecular biological kits for DNA-purification (Zymoclean DNA Clean & Concentrator Kit), Agarose gel-extraction (Zymoclean Ge/DNA Recovery Kit) and plasmid isolation (Zyppy™ Pilasmid Miniprep Ki6) were purchased from ZymoResearch (Irvine, US).

TABLE 1
List of Buffers & Media

| Buffer | Ingredients |
|---|---|
| 10x phosphate buffer | 0.17M KH₂PO₄, 0.72M K₂HPO₄, pH = 7.4 |
| (KPᵢ-buffer) | |
| Whole cell buffer | 100 mM Citric acid, 0.1% SDS, pH = 6.0 |
| Cyclodextrin (CD) buffer | 0.2% SDS, 10 mM (2-Hydroxypropyl)- |
| | β-cyclodextrin, pH = 6.0 |
| Lysis buffer | 200 mM Citric acid, 0.1% EDTA, pH = 6.0 |
| Extraction buffer | 100 mM Citric acid, 1% CHAPS, pH = 6.0 |

TABLE 2
List of media used

| Medium | Ingredients |
|---|---|
| Lysogeny broth | 10 g/L tryptone, 10 g/L NaCl, 5 g/L |
| | yeast extract |
| Auto-induction medium*(T-DAB) | 12 g/L tryptone, 24 g/L yeast extract, |
| | 2.9 g/L glucose, 11.1 g/L Glycerol, |
| | 7.6 g/L Lactose |

based on: https://www.tci.uni-hannover.de/uploads/tx_tkpublikationen/Poster_for_Wien_autoinduction_Z haopeng_Li.pdf

TABLE 3
List of primers used in this work

| | Name | |
|---|---|---|
| No | (SEQ-ID No.) | Sequence (5′→3′) Forward/Reverse |
| 1 | AacSHC_G600C | CCGTATTATACCGGCACCTGCTTCCCGGGCG/ |
| | (ID 36, ID 37) | CGCCCGGGAAGCAGGTGCCGGTATAATACGG |
| 2 | AacSHC_G600D | CCGTATTATACCGGCACCGATTTCCCGGGCG/ |
| | (ID 38, ID 39) | CGCCCGGGAAATCGGTGCCGGTATAATACGG |
| 3 | AacSHC_G600E | CCGTATTATACCGGCACCGAATTCCCGGGCG/ |
| | (ID 40, ID 41) | CGCCCGGGAATTCGGTGCCGGTATAATACGG |
| 4 | AacSHC_G600H | CCGTATTATACCGGCACCCATTTCCCGGGCG/ |
| | (ID 42, ID 43) | CGCCCGGGAAATGGGTGCCGGTATAATACGG |
| 5 | AacSHC_G600I | CCGTATTATACCGGCACCATTTTCCCGGGCG/ |
| | (ID 44, ID 45) | CGCCCGGGAAAATGGTGCCGGTATAATACGG |
| 6 | AacSHC_ G600K | CCGTATTATACCGGCACCAAATTCCCGGGCG/ |
| | (ID 46, ID 47) | CGCCCGGGAATTTGGTGCCGGTATAATACGG |
| 7 | AacSHC_G600M | CCGTATTATACCGGCACCATGTTCCCGGGCG/ |
| | (ID 48, ID 49) | CGCCCGGGAACATGGTGCCGGTATAATACGG |
| 8 | AacSHC_G600N | CCGTATTATACCGGCACCAACTTCCCGGGCG/ |
| | (ID 50, ID 51) | CGCCCGGGAAGTTGGTGCCGGTATAATACGG |
| 9 | AacSHC_G600P | CCGTATTATACCGGCACCCCGTTCCCGGGCG/ |
| | (ID 52, ID 53) | CGCCCGGGAACGGGGTGCCGGTATAATACGG |
| 10 | AacSHC_G600Q | CCGTATTATACCGGCACCCAGTTCCCGGGCG/ |
| | (ID 54, ID 55) | CGCCCGGGAACTGGGTGCCGGTATAATACGG |
| 11 | AacSHC_ G600L | CCGTATTATACCGGCACCCTATTCCCGGGCG/ |
| | (ID 56, ID 57) | CGCCCGGGAATAGGGTGCCGGTATAATACGG |
| 12 | AacSHC_G600S | CCGTATTATACCGGCACCTCGTTCCCGGGCG/ |
| | (ID 58, ID 59) | CGCCCGGGAACGAGGTGCCGGTATAATACGG |
| 13 | AacSHC_G600T | CCGTATTATACCGGCACCACCTTCCCGGGCG/ |
| | (ID 60, ID 61) | CGCCCGGGAAGGTGGTGCCGGTATAATACGG |
| 14 | AacSHC_G600V | CCGTATTATACCGGCACCGTGTTCCCGGGCG/ |
| | (ID 62, ID 63) | CGCCCGGGAACACGGTGCCGGTATAATACGG |
| 15 | AacSHC_G600Y | CCGTATTATACCGGCACCTATTTCCCGGGCG/ |
| | (ID 64, ID 65) | CGCCCGGGAAATAGGTGCCGGTATAATACGG |
| 16 | AacNMC_Y609F | GGCGATTTTTATGCGGGCTTTACCATGTATC |
| | (ID 66, ID 67) | GCCATGTG/ |
| | | CACATGGCGATACATGGTAAAGCCCGCATAAAA |
| | | ATCGC |
| 17 | AacSHC_Y420F | CAACGGCGGCTGGGGCGCGTTTGATGTGGATA |
| | (ID 68, ID 69) | ACACCAGC/ |
| | | GCTGGTGTTATCCACATCAAACGCGCCCCAGC |
| | | CGCCGTTG |
| | AacSHC_L607M | GGTTCCCGGGCGATTTTTATGCCATGTATACCA |
| | (ID 70, ID 71) | TGTATCGCC/ |
| | | GGCGATACATGGTATAGCCGGCATAAAAATCGC |
| | | CCGGGAACC |
| | AacSHC_L607S | GGTTCCCGGGCGATTTTTATAGCCATTATACCAT |
| | (ID 72, ID 73) | GTATCGCC/ |
| | | GGCGATACATGGTATAGCCGCTATAAAAATCGCC |
| | | CGGGAACC |
| | AacSHC_L607Y | GGTTCCCGGGCGATTTTTATTATGGCTATACCATG |
| | (ID 74, ID 75) | TATCGCC/ |
| | | GGCGATACATGGTATAGCCATAATAAAAATCGCCC |
| | | GGGAACC |
| | AacSHC_L607V | GGTTCCCGGGCGATTTTTGTCCCATGTATACCATG |
| | (ID 76, ID 77) | TATCGCC/ |
| | | GGCGATACATGGTATAGCCGGGACAAAAATCGCC |
| | | CGGGAACC |
| 18 | AacSHC_L607RVT | GGTTCCCGGGCGATTTTTATRNTGGCTATACCATG |
| | (ID 78, ID 79) | TATCGC/ |
| | | ATAAAAATCGCCCGGGAACCCGGTGCCGGTATAA |
| | | TACGG |
| 19 | AacSHC_1261X | GCGATGGCAGCTGGGGCGGCNDTCAGCCGCCGTG |
| | (ID 80, ID 81, | GTTTTATGC/ |
| | ID 82, ID 83) | GCGATGGCAGCTGGGGCGGCVHGCAGCCGCCGTG |
| | | GTTTTATGC/ |
| | | GCGATGGCAGCTGGGGCGGCTGGCAGCCGCCGTG |
| | | GTTTTATGC/ |
| | | TGCCGCCCCAGCTGCCATCGCCCGCCTGGCGTTCCAG |
| 20 | AacSHC_F365X | GAAACCGGGCGGCTTTGCGNDTCAGTTTGATAACGTG |
| | (ID 84, ID 85, | TATTATCCGG/ |
| | ID 86, ID 87) | GAAACCGGGCGGCTTTGCGVHGCAGTTTGATAACGTG |
| | | TATTATCCGG/ |
| | | GAAACCGGGCGGCTTTGCGTGGCAGTTTGATAACGTG |
| | | TATTATCCGG/ |
| | | CGCAAAGCCGCCCGGTTTCAGGTTCGGGCGTTTCAC |
| 21 | AacSHC_L36X | GGCTATTGGTGGGGCCCGNDTCTGAGCAACGTGAC |
| | (ID 88, ID 89, | CATG/ |
| | ID 90, ID 91) | GGCTATTGGTGGGGCCCGVHGCTGAGCAACGTGACC |
| | | ATG/ |
| | | GGCTATTGGTGGGGCCCGTGGCTGAGCAACGTGAC |
| | | CATG/ |
| | | CGGGCCCCACCAATAGCCTTCATCTTTCTGGCAGC |
| | | TCAG |
| 22 | AacSHC_S307X | GGCTGGATGTTTCAGGCGNDTATTAGCCCGGTGT |
| | (ID 92, ID 93, | GGG/ |
| | ID 94, ID 95) | GGCTGGATGTTTCAGGCGVHGATTAGCCCGGTGT |
| | | GGG/ |
| | | GGCTGGATGTTTCAGGCGTGGATTAGCCCGGTGT |
| | | GGG/ |
| | | CGCCTGAAACATCCAGCCGCCATAATCCAGTTCCACG |
| 23 | AacSHC_A306X | GGCGGCTGGATGTTTCAGNDTAGCATTAGCCCGGTG/ |
| | (ID 96, ID 97, | GGCGGCTGGATGTTTCAGVHGAGCATTAGCCCGGTG/ |
| | ID 98, ID 99) | GGCGGCTGGATGTTTCAGTGGAGCATTAGCCCGGTG/ |
| | | CTGAAACATCCAGCCGCCATAATCCAGTTCCACGCCA |
| | | TACAG |

ID represents the SEQ-ID No. in the sequence listing.

General Analytics

Nuclear Magnetic Resonance

¹H- und ¹³C-NMR spectra were recorded on a BrukerAvance 500 Spectrometer at 500.15 MHz for ¹H- and 125 MHz for ¹³C. The chemical shifts δ are referred to tetramethylsilane (=TMS) in ppm set to 0. All substances were dissolved in CDCl₃ and recorded at room temperature.

Circular Dichroism

The specific optical rotation of the compounds was measured on a Perkin Elmer Polarimeter 241. Therefore the substance was dissolved in CHCl₃ (c=0.5 mg/ml) and the specific rotation was measured with a sodium and a mercury spectral lamp.

Gas Chromatography

GC analyses were performed using an Agilent GC 7820A equipped with a mass spectrometer MSD 5977B and a HP-5MS capillary column (Agilent, 30 m×250 μm×0.25 μm) and helium as carrier gas with a constant pressure of 14.168 ψ. Injections (1 μL) were performed in split mode (10:1). Relative conversion rates were calculated directly from GC-MS spectra by integration-quotient of substrates and products. Chiral GO analysis was performed on a Shimadzu GC-2010 equipped with a OP ChiraSil-Dex GB capillary column (Agilent, 25 m×250 μm×0.25 μm) and hydrogen as carrier gas with constant velocity (linear velocity: 33.1 cm/s). Injections (1 μL) were performed in split mode (5:1). Temperature programs are listed in table 4.

TABLE 4
Temperature programs used in this work

| Name | Rate (° C./min) | Temp. (° C.) | Hold (min) |
|---|---|---|---|
| Dihydroion long | | 120 | 0.1 |
| | 2 | 145 | 0.6 |
| Dihydrion short | | 120 | 0.1 |
| | 2 | 137 | 0.6 |
| Calmusal | | 110 | 0.1 |
| | 2 | 135 | 0.6 |
| General | | 50 | 3 |
| | 6 | 120 | 0 |
| | 10 | 150 | 0 |
| | 15 | 170 | 0 |
| | 20 | 200 | 0 |
| | 25 | 250 | 0 |
| | 30 | 310 | |
| Chiral | | 70 | 3 |
| | | 140 | 0 |
| | 8 | 180 | 2 |

Chemical Synthesis

Synthesis of Geranyl Isopropanol

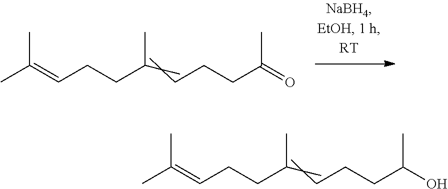 [SMILES: CC(=O)CCCC(C)(C)C(C)CCC=C(C)C ; CC(C)=CCCC(C)C(C)(C)CCCC(C)O]

For the reduction reaction geranyl acetone (0.50 ml, 2.34 mmol 1.00 eq.) was dissolved in ethanol (10 ml). Sodium borohydride (0.088 g, 2.34 mmol, 1.00 eq.) was then added carefully and the reaction mixture was stirred at room temperature for 1 h. After the reaction was complete, the mixture was quenched with 0.5 N HCl (2 ml) and stirred again for 30 min. Then distilled water (50 ml) was added and the aqueous phase was extracted three times with DCM. The combined organic phases were dried over CaCl₂ and the geranyl isopropanol was obtained as a clear oil (0.49 ml, 2.04 mmol, 87%).

¹H-NMR (CDCl₃, 500 MHz): δ (ppm) 1.19 (d, J=2.9 Hz, 3H), 1.50 (quart, J=7.7 Hz, 2H) 1.6 (s, 3H), 1.62 (s, 3H), 1.68 (s, 3H), 1.88-1.92 (t, J=7.3 Hz, 2H), 2.04-2.12 (m, 4H), 3.77-3.84 (sept, J=17.43 Hz, 1H), 5.05-5.10 (t, J=6.7 Hz, 1H), 5.12-5.17 (t, J=6.8 Hz, 1H). ¹³C-NMR (CDCl₃, 125 MHz): δ (ppm) 16.50 (1C), 16.66 (1C), 22.44-25.63 (4C), 38.15-38.70 (2C), 66.97 (1C), 75.67 (1C), 122.22-123.24 (2C), 134.9 (1C). MS (EI): m/z (%)=196 (0.3), 153 (32), 135 (21), 109 (58), 95 (21), 82 (19), 81 (21), 69 (100), 68 (13), 67 (44). The data are consistent with the literature¹.

### Synthesis of 6,10-dimethylundeca-5,9-dien-2-ol (23)

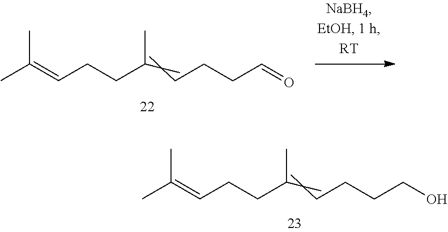 [SMILES: CC(C)=CCCC(C)C(C)(C)CCCC=O ; CC(C)=CCCC(C)C(C)(C)CCCCO]

The reaction was carried out analog to synthesis (1). The product was obtained as a clear oil (0.21 ml, 1.04 mmol, 43%).

¹H-NMR (CDCl₃, 500 MHz): δ (ppm) 1.19-1.34 (m, 2H), 1.55-1.59 (m, 2H) 1.61 (s, 3H), 1.62-1.65 (m, 1H), 1.68 (s, 3H), 1.69-1.71 (m, 2H), 2.04-2.12 (m, 4H), 3.62-3.67 (t, J=6.6 Hz, 2H), 5.03-5.19 (m, 2H). The data is consistent with the literature².

Sulfuric Acid Catalyzed Cyclization of (−)-γ-Dihydroionone

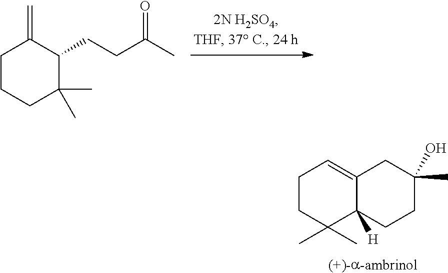

For the cyclization reaction (−)-γ-dihydroionone (400 μL, 1.8 mmol) was dissolved in THE (15 mL) in a 50 mL Schott-bottle. 2N sulfuric acid (5 mL) was then added and the reaction mixture was shaken at 37° C. for 24 h. The reaction was quenched by addition of water (20 mL) and extracted with Diethylether (3×30 mL). The combined organic phases were dried over MgSO₄ and purified via silica chromatography (10:1, hexane: ethyl acetate) to yield the slightly yellowish liquid (+)-α-ambrinol (350 μL, 1.5 mmol, 88% yield); ([α]_{D}²⁰=+84.6; Lit.=81.8³).

¹H-NMR (CDCl₃, 500 MHz): δ (ppm) 0.87 (s, 3H), 0.91 (s, 3H) 1.14 (m, 1H), 1.22 (s, 3H), 1.24-1.40 (m, 3H), 1.45-1.51 (m, 2H), 1.67-1.74 (m, 2H), 1.98-2.02 (m, 2H), 2.06-2.17 (m, 2H), 5.45 (t, J=3.84 Hz, 1H). ¹³C-NMR (CDCl₃, 125 MHz): δ (ppm) 22.6 (1C), 23.8 (1C), 25.05 (1C), 26.02 (1C), 28.07 (1C), 29.24 (1C), 31.11 (1C), 28.95 (1C), 47.25 (1C), 49.82 (1C), 70.28 (1C), 122.04 (1C), 137.39 (1C). The data is consistent with the literature⁴.

(+)-α-ambrinol MS (EI): m/z (%)=194 (5), 176 (40), 161 (30), 136 (100), 121 (66), 120 (40), 109 (28), 105 (31), 95 (49), 93 (28).

Side product: (−)-β-ambrinol: MS (EI): m/z (%)=194 (6), 176 (55), 161 (100), 136 (40), 121 (84), 107 (43), 106 (46), 105 (60), 93 (52), 91 (42).

General Methods

Plasmid Isolation

Isolation of the plasmid proceeded following to the standard protocol of Zyppy™ Plasmid Miniprep Kit by ZymoResearch.⁵ For the photometric determination of the plasmid DNA concentration, 1 μL was measured on a Nanodrop 1000 (Agilent, Santa Clara, US) at a wavelength of 260 nm.

Site-Saturation/-Directed Mutagenesis

The gene encoding for AacSHC (UniProt: P33247) or a variant based on this gene was cloned into a pET-22b(+) vector system (Merck, Darmstadt, Germany). SacI and NdeI were used as restriction sites. Cloning followed the standard protocol of Novagene's KOD Hot Start DNA Polymerase.⁶ The composition of the PCR mixture and the temperature profile are described in Table 5 and Table 6.

TABLE 5
Composition of the PCR mixture

| substance | volume [μl] | final concentration | |
|---|---|---|---|
| daH₂0 | 29 | | |
| DMSO | 2.5 | | |
| KOD Hot Start Buffer (10x) | 5 | 1x | |
| dNTPs (2 mM each) | 5 | 250 | μm (each) |
| MgSO₄ (25 mM) | 4.5 | 2 | mM |
| Template DNA | 1 | 0.5-5 | ng/μl |
| Primer forward (10 μM) | 1 | 0.2 | μm |
| Primer reverse (10 μM) | 1 | 0.2 | μm |

| | |
|---|---|
| KOD Hot Start DNA Polymerase | 1 |

TABLE 6
PCR temperature profile

| step | Temperature [° C.] | time [s] | cycles |
|---|---|---|---|
| Initial denaturation | 95 | 120 | 1 |
| Denature | 95 | 30 | |
| Annealing | 60 | 30 | 30 |
| Extension | 70 | 210 | |
| Final extension | 72 | 420 | 1 |

Site-saturation libraries were generated employing the “22c-trick” method.⁷ PCR products were digested with 1 μL DpnI for 2 h at 37° C., purified by agarose gel electrophoresis and ligated into the pET22b(+) vector by Gibson assembly⁸. After purification using the DNA Clean & Concentrator™-5 kit⁹ the plasmids were transformed via heat-shock method. Site-directed clones were digested and directly transformed afterwards.

Plasmid Transformation

Chemically competent cells based on rubidium chloride were produced for the transformation of the plasmid DNA.¹⁰ The transformation was carried out under sterile conditions. For site saturation libraries 3 μL of the purified PCR product was added to 25 μL XL1-blue competent cells and incubated for 30 min on ice, followed by a heat shock at 42° C. for 105 s with subsequent ice cooling for 3 min. After adding 500 μl of LB medium, the cells were incubated for 40 min at 37° C. and used for inoculation of a 5 mL LB medium (Ampicillin, c_{end}=100 μg/ml) pre-culture overnight. After isolation of the plasmid, transformation into 50 μL BL21 (DE3) was performed using the heat shock method. After regeneration 150 μL were streaked out on an agar plate (Ampicillin, c_{end}=100 μg/ml) and incubated at 37° C. overnight. For quality control the plasmid was isolated from another 150 μL and sent for sequencing. For site-directed mutants the PCR product was directly transformed into XL1-blue competent cells after digest. After regeneration 300 μL were streaked out on an agar plate for single clone picking.

Expression of AacSHC Libraries in 96-DW Plates

Individual colonies were picked from generated agar plates and cultivated in 500 μL LB medium (Ampicillin, c_{end}=100 μg/ml) for 18-20 h at 37° C., 800 rpm. Expression cultures were inoculated with 10 μL of the pre-culture into 1 mL of T-DAB autoinduction medium (Ampicillin, c_{end}=100 μg/ml) with lactose as the inductor. The cultures were incubated for 20 h at 37° C., 800 rpm and harvested afterwards (4000×g, 20 min).

Expression in 24 DW-Plates

Individual colonies were picked from generated agar plates and cultivated in 2 mL LB medium (Ampicillin, c_{end}=100 μg/ml) for 18-20 h at 37° C., 180 rpm. Expression cultures were inoculated with 40 μL of the pre-culture into 4 mL of T-DAB autoinduction medium (Ampicillin, c_{end}=100 μg/ml) with lactose as the inductor. The cultures were incubated for 20 h at 37° C., 600 rpm and harvested afterwards (4000×g, 20 min).

Thermolysis Purification^{11,12}

Harvested or lyophilized cells were resuspended in 1 mL Lysis buffer and incubated for 60 min at 70° C. The cell suspension was centrifuged (14000×g, 1 min) and the supernatant was discarded. As the enzyme is membrane-bound 1 mL 1%-CHAPS buffer was added to extract it from the cell pellet by shaking at room temperature for 1d, 600 rpm. After subsequent centrifugation (14000×g, 1 min) the supernatant containing the AacSHC was transferred to a new tube followed by SDS-PAGE analysis and determination of enzyme concentration by using the Nanodrop 1000 (Agilent, Santa Clara, US). Therefore the “Protein A280” mode was chosen with MW=71439 Da and molar extinction coeffizient ε=185180 as protein specific data.

SDS-PAGE

After protein purification and extraction 20 μl of the enzyme preparation was mixed with 10 μl SDS loading buffer and heated to 95° for 10 min. Afterwards 10 μl of the preparation were loaded on the pre-prepared SDS-PAGE.

Screening of AacSHC Libraries Via GC-MS

Harvested pellets were resuspended in 400 μL whole cell buffer and transferred to another 96-DW plate equipped with 1.2 mL glass inlets. Afterwards 4 μL substrate/DMSO stock solution (substrate c_{end}=2 mM) was added directly into the cell suspension, the plates were sealed and shaken for 20 h at 30° C., 600 rpm. In order to stop the reaction 600 μL cyclohexene/o-xylol (1:1) was added and the mixture was incubated for 10 min. The plates were centrifuged (4000×g, 5 min), sealed using PP-sealings and a GC-MS equipped with a PAL-Sampler was used to inject directly from the organic phase. Quantification was made directly from the Total Ion Count chromatogram by quotient AREA_{product}/(AREA_{substrate}+AREA_{product})*100. In total 90 variants per plate were screened. Promising variants were rescreened by expression in 24 DW-plates.

Verification of Promising Hits

Promising candidates from the 96-DW screening were taken for inoculation of a 5 mL LB pre-culture. Afterwards the plasmids were isolated and transformed for single colony picking. The single colonies were expressed in 24 DW-plates and after harvesting the OD₆₀₀ was set to 20 in whole cell buffer substrate was added (c_{end}=4.4 mM) The reactions were carried out at least in technical duplicates. Reactions were stopped by adding Dichloromethane. After two extraction the resulting organic phase was measured directly over GC-MS. Quantification was made directly from the Total Ion Count chromatogram by quotient AREA_{product}/(AREA_{substrate}+AREA_{product})*100.

Determination of Total Turnover Number

After expression and harvesting in 24 DW-plates the cell pellets were frozen at −80° C. overnight. Afterwards the frozen pellets were lyophilized in a Christ alpha2-4LD plus overnight. For the reaction setup 10 mg of the E. coli whole cells were resuspended in 1 ml cyclodextrin buffer and 2 μl (c_{end}=8.8 mM) of substrate was added to the suspension and the reaction was stirred for 20 h at 30° C. The reaction was stopped by addition of DCM and 10 mM of 1-Undecanol was added. The reaction was extracted three times and the combined organic phases were measured over GC-MS. Quantification was made by 1-Undecanol as internal standard. The protein concentration was determined by extracting the enzyme from 10 mg for each batch in triplikates via thermolysis (see (6)). Verification and quality control was done by SDS-PAGE.

Up-Scaling Reactions

In order to isolate and determine the structure of the products upscalings of the biotransformations were performed. Therefore, the corresponding variant was expressed and the harvested cell pellets were lyophilized. Afterwards 3 g of lyophilized whole cells were resuspended in 200 mL buffer (0.1% SDS, 50 mM Citric acid, 5 mM (2-Hydroxypropyl)-β-cyclodextrin) and 200 μl substrate was added. The reactions were carried out in closed 250 mL flasks at 30° C. and 250 rpm for seven days. The crude product was centrifuged to get rid of the cell debris. The aqueous phase containing the product encapsulated by cyclodextrin was extracted with diethyl ether three times, reduced under vacuum, purified over column chromatography (petroleum ether: ethyl acetate; 50:1->10:1) and evaluated via NMR and GC/MS.

For Z-geranyl acetone conversion 10 g of lyophilized whole cells were used in 500 mL cyclodextrin (CD) buffer and 2 g (2.24 ml) substrate was added.

E-geranyl acetone 16t with G600R (SEQ-ID No.16)

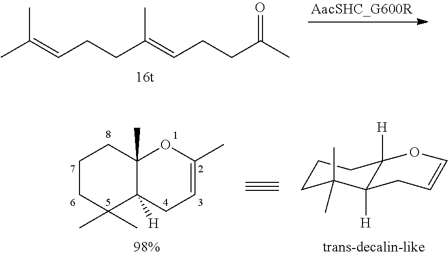

Colorless oil, 0.167 ml, 0.77 mmol, 85% yield. (4S,8S)-2,5,5,8-tetramethyl-4,5,6,7,8,8-hexahydro-4H-chromene 11t: ¹H-NMR (CDCl₃, 500 MHz): δ (ppm) 0.81 (s, 3H), 0.91 (s, 3H) 1.17 (s, 3H), 1.21-1.29 (m, 1H), 1.4-1.6 (m, 5H), 1.68 (s, 3H), 1.72-1.94 (m, 3H), 4.4-4.5 (m, 1H). ¹³C-NMR (CDCl₃, 125 MHz): δ (ppm) 19.07 (1C), 19.21 (1C), 19.82 (1C), 20.51 (1C), 20.77 (1C) 30.31 (1C), 32.25 (1C), 39.99 (1C), 41.65 (1C), 48.37 (1C), 76.48 (1C), 94.97 (1C), 147.97 (1C). The data is consistent with the literature.¹³

Z-geranyl acetone 16c with G600R (SEQ-ID No.16)

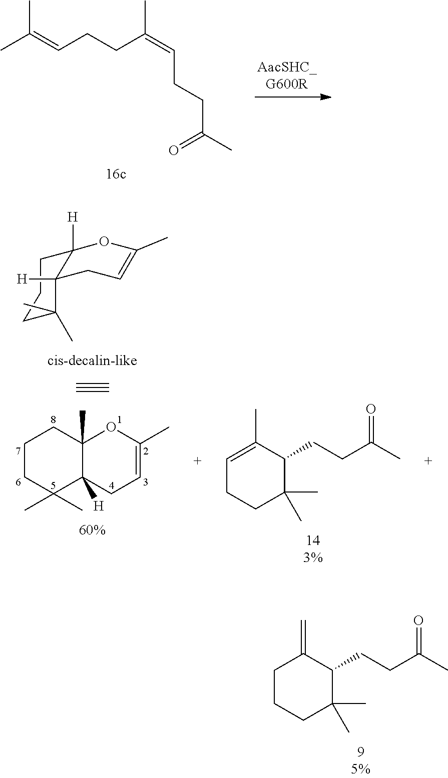

Colorless oil, 0.098 ml, 0.45 mmol, 49% yield. (4R,8S)-2,5,5,8-tetramethyl-4,5,6,7,8,8-hexahydro-4H-chromene 11t: ¹H-NMR (CDCl₃, 500 MHz): δ (ppm) 0.85 (s, 3H), 0.87 (s, 3H) 1.16 (s, 3H), 1.32-1.39 (m, 1H), 1.54 (s, 3H), 1.6-1.66 (m, 3H), 1.68 (s, 3H), 1.72-1.97 (m, 3H), 2.14-2.27 (m, 1H), 4.4-4.5 (d, J=2.6 Hz, 1H). ¹³C-NMR (CDCl₃, 125 MHz): δ (ppm) 18.13 (1C), 19.79 (1C), 20.54 (1C), 21.19 (1C), 26.50 (1C) 32.46 (1C), 33.73 (1C), 39.66 (1C), 41.99 (1C), 44.00 (1C), 74.71 (1C), 94.56 (1C), 148.76 (1C).

Z-geranyl acetone 16c with NMC

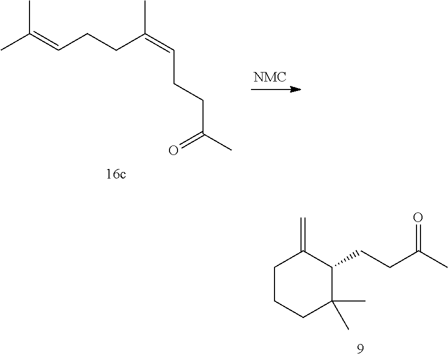

Colorless oil, 1.97 ml, 9.1 mmol, 89% yield. (−)-γ-dihydroionone 9: ¹H-NMR (CDCl₃, 500 MHz): δ (ppm) 0.87 (s, 3H), 0.92 (s, 3H) 1.10-1.30 (m, 2H), 1.42-1.62 (m, 2H), 1.66-1.70 (m, 1H), 1.76-1.83 (m, 1H), 1.97-2.04 (m, 2H), 2.11 (s, 3H), 2.22-2.45 (m, 2H), 4.50-4.51 (d, J=1.03 Hz, 1H), 4.75-4.77 (m, 1H). ¹³C-NMR (CDCl₃, 125 MHz): δ (ppm) 20.31 (1C), 22.62 (1C), 23.52 (1C), 26.5 (1C), 28.3 (1C), 30.20 (1C), 32.00 (1C), 34.83 (1C), 42.38 (1C), 53.40 (1C), 109.5 (1C), 149.09 (1C), 209.52 (1C). The data is consistent with the literature.¹⁴

E/Z-geranyl isopropanol 21 with G600N/L607S (SEQ-ID No.17)

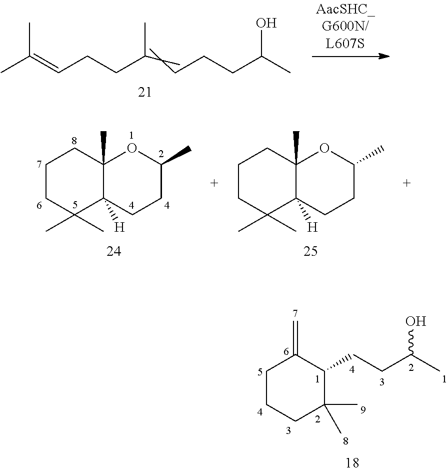 [SMILES: CC(C)=CCCC(C)C(C)(C)CCCC(C)O ; C[C@H]1CC[C@H]2C(C)(C)CCC[C@]2(C)O1 ; C[C@@H]1CC[C@H]2C(C)(C)CCC[C@]2(C)O1 ; C=C1CCCC(C)(C)[C@H]1CCC(C)O]

Yellowish oil, 0.020 ml, 0.9 mmol, 10% yield. ¹H-NMR (CDCl₃, 500 MHz): δ (ppm) 2S,4S,8S-Tetrahydroedulane 24: 0.81 (s, 3H), 0.89 (s, 3H), 1.14-1.15 (d, J=3.1, 3H), 1.23 (s, 3H), 1.28 (s, 1H) 1.33 (s, 1H), 1.42-1.53 (m, 5H), 1.56 (s, 2H), 1.62-1.77 (m, 4H), 3.97-4.04 (m, 1H). 2R,4S,8S-Tetrahydroedulane 25: 0.74 (s, 3H), 0.87 (s, 3H), 1.09-1.10 (d, J=3.2, 3H), 1.23 (s, 3H), 1.28 (s, 1H) 1.33 (s, 1H), 1.42-1.53 (m, 5H), 1.56 (s, 2H), 1.62-1.77 (m, 4H), 3.72-3.79 (m, 1H). ¹³C-NMR (CDCl₃, 125 MHz): 2R,4S,8S-Tetrahydroedulane 25: δ (ppm) 19.54 (1C), 19.59 (1C), 20.19 (1C), 20.78 (1C), 22.72 (1C), 32.11 (1C), 33.37 (1C), 35.61 (1C), 40.75 (1C), 41.67 (1C), 53.30 (1C), 65.51 (1C), 74.83 (1C). The data is consistent with the literature.¹⁵

4-((R)-2,2-dimethyl-6-methylenecyclohexyl)butan-2-ol 18: Characteristic methylene signals at ¹H-NMR (CDCl₃, 500 MHz): δ (ppm) 4.53 (d, J=1.25 Hz, 1H) and 4.75 (t, J=1.25 Hz, 1H). From the chiral GC data and the enantiopure monocyclization of 16c we assume the stereocenter here to be R.

### 6,10-dimethylundeca-5,9-dien-2-ol 23 with G600N/L607S (SEQ-ID No.17)

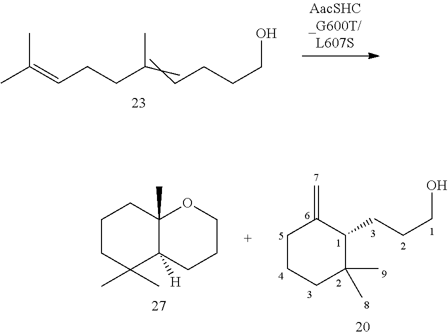 [SMILES: CC(C)=CCCC(C)C(C)(C)CCCCO ; CC1(C)CCC[C@]2(C)OCCC[C@@H]12 ; C=C1CCCC(C)(C)[C@H]1CCCO]

Characteristic C7-methylene signals for 20 at ¹H-NMR (CDCl₃, 500 MHz): 4.55 (d, J=1.00 Hz, 1H) and 4.75 (t, J=1.30 Hz, 1H). From the chiral GC data and the enantiopure monocyclization of 10c we assume the stereocenter here to be R.

Table 7 shows the relative conversion rates in % of substrate mixture 16 and isolated 16t and 16c with the wild-type enzyme and the variant G600R and the corresponding product selectivities.

| | | (E/Z)-GER | (E)-GER | (Z)-GER |
|---|---|---|---|---|
| | | 16 | 16t | 16c |
| WT | | 23.2 | 29.0 | 0.7 |
| G600R | | 80.4 | 95.7 | 68 |
| error WT | | 4.1 | 2.5 | 0.2 |
| error G600R | | 5.1 | 0.8 | 4.3 |
| selectivity WT | bicyclic | 100 | 100 | 60 |
| selectivity | bicyclic | 95 | 100 | 85 |
| G600R | monocyclic | 5 | | 15 |

Reaction conditions: E. coli whole cells harboring AacSHC variant resuspended in whole-cell buffer (0.1 M citric acid, 0.1% SDS, pH=6.0) with an OD₆₀₀=20, 20 h, 30° C., 4.4 mM substrate (=1 μl in 1 ml cell suspension).

TABLE 8
Relative conversion rates in % of the substrate
Z-geranyl acetone X with all variants at position
600 and the corresponding product selectivities

| | Hexahydrochromene | 9 | 14 | overall conv. | error |
|---|---|---|---|---|---|
| G600R (ID 16) | 26.2 | 1.7 | 0.7 | 28.5 | 0.6 |
| G600M (ID 18) | 25.5 | 0.7 | 0.6 | 26.7 | 1.1 |
| G600T (ID8) | 8.8 | 7.9 | 3.8 | 20.4 | 0.4 |
| G600L (ID 19) | 16.1 | 0.4 | 0.3 | 16.8 | 0.3 |
| G600N (ID 11) | 9.9 | 1.0 | 1.6 | 12.4 | 1.2 |
| G600Q (ID13) | 9.6 | 0.4 | 0.5 | 10.4 | 2.0 |
| G600Y (ID20) | 8.1 | 0.6 | 0.3 | 9.0 | 0.2 |
| G600C (ID15) | 6.8 | 1.5 | 0.7 | 9.0 | 0.4 |
| G600S (ID10) | 4.6 | 2.7 | 1.2 | 8.5 | 0.5 |
| G600K (ID21) | 6.6 | 0.7 | 0.3 | 7.5 | 1.2 |
| G600D (ID12) | 5.4 | 1.2 | 0.7 | 7.3 | 2.5 |
| G600E (ID14) | 5.8 | 1.1 | 0.3 | 7.2 | 1.8 |
| G600V (ID22) | 5.4 | 0.2 | 0.1 | 5.7 | 2.3 |
| G600A (ID9) | 2.9 | 0.9 | 0.3 | 4.2 | 1.0 |
| G600F (ID23) | 3.4 | 0.3 | 0.1 | 3.8 | 2.1 |
| G600I (ID24) | 3.4 | 0.1 | 0.0 | 3.6 | 0.5 |
| G600W (ID25) | 2.6 | 0.4 | 0.2 | 3.1 | 0.3 |
| G600H (ID26) | 1.1 | 0.1 | 0.1 | 1.3 | 0.8 |
| WT (ID1) | 0.5 | 0.1 | 0.1 | 0.7 | 0.4 |
| G600P (ID 27) | 0.1 | 0.0 | 0.0 | 0.1 | 0.0 |

ID represents the SEQ-ID No. in the sequence listing.

Reaction conditions: E. coli whole cells harboring AacSHC variant resuspended in whole-cell buffer (0.1 M citric acid, 0.1% SDS, pH=6.0) with an OD₆₀₀=22, 20 h, 30° C., 8.48 mM substrate (=2 μl in 1 ml cell suspension).

TABLE 9
Relative conversion rates in % of the substrate Z-
geranyl acetone X with the variants at position 607
and the corresponding product selectivities.

| | Hexahydrochromene | 9 | 14 | overall conv. | error |
|---|---|---|---|---|---|
| L607S (ID28) | 8.8 | 6.3 | 1.8 | 16.9 | 1.2 |
| L607M (ID29) | 10.5 | 0.4 | 0.3 | 11.2 | 0.5 |
| L607A (ID30) | 5.5 | 3.3 | 0.8 | 9.5 | 0.6 |
| L607V (ID31) | 3.8 | 1.8 | 0.5 | 6.1 | 0.7 |
| L607G (ID32) | 1.2 | 0.4 | 0.2 | 1.7 | 0.2 |

ID represents the SEQ-ID No. in the sequence listing.

Reaction conditions: E. coli whole cells harboring AacSHC variant resuspended in whole-cell buffer (0.1 M citric acid, 0.1% SDS, pH=6.0) with an OD₆₀₀=20, 20 h, 30° C., 8.8 mM substrate (=2 μl in 1 ml cell suspension).

TABLE 10
Relative conversion rates in %, corresponding selectivities and total turnover
numbers (TTN) of the wild-type enzyme and the engineered enzymes.

| Enzymeconc. | 1.54 | 1.32 | 1.5 | 1.54 | 1.64 | 1.38 |
|---|---|---|---|---|---|---|
| in g/l | | | | | | |
| Enzymeconc. | 2.15569E−05 | 1.848E−05 | 2.1E−05 | 2.156E−05 | 2.296E−05 | 1.932E−05 |
| in mol/l | | | | | | |
| MW = 71439 | WT | G600R | G600T | N1 | N2 | NMC |
| g/mol | | | | (+L607A) | (+Y420F) | (+306V) |
| chromene | 0.5 | 21.9 | 6.4 | 4.4 | 1.8 | 1.0 |
| 9 | 0.1 | 1.5 | 7.4 | 22.2 | 63.8 | 95.2 |
| 14 | 0.1 | 0.7 | 1.1 | 1.4 | 2.2 | 2.1 |
| overall conversion | 0.7 | 24.1 | 14.9 | 28.0 | 67.8 | 98.3 |
| error conversion | 0.2 | 3.0 | 0.5 | 3.2 | 0.9 | 2.4 |
| TTN | 2.9 | 114.8 | 62.4 | 114.4 | 260.0 | 447.8 |
| error TTN | 0.8 | 14.3 | 2.1 | 13.1 | 3.4 | 10.9 |

10 mg lyophilized E. coli whole cells harboring AacSHC variant (18-22 μM) resuspended in 1 mL whole-cell buffer (0.1 M citric acid, 0.1% SDS, 10 mM 2-Hydroxypropyl)-β-cyclodextrin, pH=6.0), 24 h, 30° C., 8.8 mM substrate.

TABLE 11
Relative conversion rates in % of the substrate Z-geranyl acetone
X with the variants N2, Y420F/L607A, Y420F/G600T/L607A/Y609F
and Y609F and the corresponding product selectivities

| | | | | overall | |
|---|---|---|---|---|---|
| | chromene | 9 | 14 | conv. | error |
| N2 (Y420F/G600T/L607A) | 1.4 | 58 | 1.8 | 60.2 | 5.4 |
| (ID5) | | | | | |
| Y420F/L607A (ID33) | 2.6 | 8.2 | 0.5 | 12 | 1.1 |
| Y420F/G600T/L607A/Y609F | 1 | 1 | 0.2 | 2.2 | 0.1 |
| (ID34) | | | | | |
| Y609F (ID35) | 0 | 0 | 0 | 0 | 0 |

ID represents the SEQ-ID No. in the sequence listing.

Reaction conditions: E. coli whole cells harboring AacSHC variant resuspended in whole-cell buffer (0.1 M citric acid, 0.1% SDS, pH=6.0) with an OD₆₀₀=22, 20 h, 30° C., 8.8 mM substrate (=2 μl in 1 ml cell suspension).

Biotransformation of 16 Using Other Cyclases

In the following experiment the capability of other cyclases to perform the monocyclization reaction is shown. Therefore, the thermophilic squalene-hopene cyclase from Thermesynechococcus elongatus (TeSHC) which naturally harbors a phenylalanine at position 429 (corresponding position in AacSHC Y420) was chosen. The results show 2% conversion of the substrate 16 towards monocyclic product 9, therefore, confirm the findings of the present invention and show the general capability of squalene-hopene cyclases to perform this reaction (FIGURE).

### LITERATURE

1. Li, Y., Lu, B., Li, C. & Li, Y. First total synthesis of (±) Hedaol B. Synth. Commun. 33, 1417-1423 (2003).
2. Brooks, J. L., Xu, L., Wiest, O. & Tan, D. S. Diastereoselective Synthesis of Highly Substituted Tetrahydrofurans by Pd-Catalyzed Tandem Oxidative Cyclization-Redox Relay Reactions Controlled by Intramolecular Hydrogen Bonding. J. Org. Chem. 82, 57-75 (2017).
3. Justicia, J. et al Titanium-catalyzed enantioselective synthesis of α-ambrinol. Adv. Synth. Catal 350, 571-576 (2008).
4. Serra, S. An expedient preparation of enantio-enriched ambergris odorants starting from commercial ionone alpha. Flavour Fragr. J. 28, 46-52 (2013).
5. Zymo Research. Zyppy™ Plasmid Miniprep Kit. Instr. Man. 4037, 1-9 (2014).
6. Biosystems, A., Drive, L. C., City, F. & Countries, A. O. <TB341.pdf>. 1-9 (1800).
7. Kille, S. et al. Reducing codon redundancy and screening effort of combinatorial protein libraries created by saturation mutagenesis. ACS Synth. Biol. 2, 83-92 (2013).
8. Gibson, D. G. et al. Enzymatic assembly of DNA molecules up to several hundred kilobases. Nat. Methods 6, 343-345 (2009).
9. America, S. Instruction Manual Instruction Manual. Int. Bus. 1-183 (2003).
10. Green, R. & Rogers, E. J. Chemical Transformation of E. coli Rachel. Methods 3-6 (2014) doi:10.1016/B978-0-12-418687-3.00028-8.Chemical.
11. Koschorreck, K., Wahrendorff, F., Biemann, S., Jesse, A. & Urlacher, V. B. Cell thermolysis—A simple and fast approach for isolation of bacterial laccases with potential to decolorize industrial dyes. Process Biochem. 56, 171-176 (2017).
12. Ren, X., Yu, D., Han, S. & Feng, Y. Thermolysis of recombinant Escherichia coli for recovering a thermostable enzyme. Biochem. Eng. J. 33, 94-98 (2007).
13. Seitz, M., Syron, P., Steiner, L., Klebensberger, J. & Nestl, B. M. Synthesis of Heterocyclic Terpenoids by Promiscuous Squalene—Hopene Cyclases. ChemBioChem 14, 436-439 (2013).
14. Fuganti, C., Serra, S. & Zenoni, A. Synthesis and olfactory evaluation of (+)- and (−)-γ-ionone. Helv. Chim. Acta 83, 2761-2768 (2000).
15. Linares-Palomino, P. J., Salido, S., Altarejos, J., Nogueras, M. & S??nchez, A. Synthesis and odour evaluation of stereoisomers of octahydrobenzopyran derivatives. Flavour Fragr. J. 21, 659-666 (2006).
16. Burghardt, T. E. Developments in the deprotection of thioacetals. J. Sulfur Chem. 26, 411-427 (2005).
17. Land, H. & Humble, M. S. YASARA: A tool to obtain structural guidance in biocatalytic investigations. in Methods in Molecular Biology vol. 1685 43-67 (Humana Press Inc., 2018).
18. Waterhouse, A. et al. SWISS-MODEL: Homology modelling of protein structures and complexes. Nucleic Acids Res. 46, W296-W303 (2018).
19. Chen, D. et al. Regulation of protein-ligand binding affinity by hydrogen bond pairing. Sci. Adv. 2, (2016).
20. Syrén, P. O., Hammer, S. C., Claasen, B. & Hauer, B. Entropy is key to the formation of pentacyclic terpenoids by enzyme-catalyzed polycyclization. Angew. Chemie—Int. Ed. 53, 4845-4849 (2014).

1. A mutant enzyme with Squalene-hopene cyclase activity, selected from mutants of a wild-type enzyme comprising an amino acid sequence selected from SEQ-ID Nos. 1 to 3 or an amino acid sequence derived therefrom with a degree of sequence identity in the range of from 60 to 99.9% of SEQ-ID No. 1 to 3, wherein the mutant catalyzes at least the one-step monocyclization of a substrate of general formula (I) 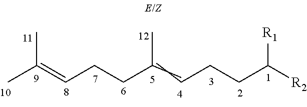
to a monocyclic compound of formula (II) 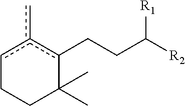
wherein at least one of substituents R¹ und R² is selected from the group consisting of ═O, —OH, thiol, amino, ester, halogen, nitro or nitrile groups and wherein at least one of substituents R¹ und R² is selected from hydrogen, alkyl or alkylene groups.

2. The mutant enzyme in accordance with claim 1, comprising
a) a mutation in position G600 of SEQ-ID No. 1 or
b) a mutation in an amino acid sequence selected from amino acid sequences SEQ-ID Nos. 2 to 3 wherein the position of the mutation corresponds to position G600 of SEQ-ID No. 1.

3. The mutant enzyme in accordance with claim 1 in which up to 10% of the amino acid groups are modified compared to SEQ-ID No. 1 or SEQ-ID No. 2 to 3 by deletion, insertion, substitution, addition or a combination thereof.

4. The mutant enzyme in accordance with claim 1 in which the mutation in position G600 of SEQ-ID No. 1 or in a position corresponding to position G600 in SEQ-ID No. 1 in one of the amino acid sequences SEQ-ID No. 2 to SEQ-ID No. 3 is a substitution selected from the group consisting of G600A, G600S, G600C, G600T, G600N, G600D, G600Q and G600E.

5. The mutant enzyme in accordance with claim 1 comprising in addition at least one mutation in one of positions Y420 or L607 of SEQ-ID No. 1 or at least one mutation in an amino acid sequence selected from amino acid sequences SEQ-ID No 2 to SEQ-ID No. 3 wherein the position of the mutation corresponds to position Y420 respectively L607 of SEQ-ID No. 1.

6. The mutant enzyme in accordance with claim 1 comprising three mutations in positions Y420, G600 und L 607 of Sequence-ID No. 1 or three mutations in an amino acid sequence selected from amino acid sequences SEQ-ID Nos 2 to 3 wherein the position of the mutation corresponds to position Y420, G600 and L607 of SEQ-ID No. 1.

7. The mutant enzyme in accordance with claim 1 comprising four mutations in positions A306, Y420, G600 und L 607 of Sequence-ID. No. 1 or four mutations in an amino acid sequence selected from amino acid sequences SEQ-ID Nos 2 to 3 wherein the position of the mutations correspond to positions A306, Y420, G600 and L607 of SEQ-ID No. 1.

8. The mutant enzyme corresponding to claim 1 comprising an amino acid sequence selected from SEQ-ID No. 4 to 8 and 34.

9. A nucleic acid sequence, encoding for an enzyme mutant in accordance with claim 1.

10. An expression cassette, comprising a nucleic acid sequence in accordance with claim 9.

11. A recombinant vector, comprising under the control of at least one regulative element, at least one nucleic acid sequence in accordance with claim 9.

12. A recombinant microorganism comprising at least one nucleic acid sequence in accordance with claim 9.

13. A process for the manufacture of compounds of formula (II) 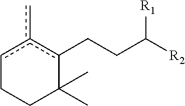
wherein at least one of substituents R¹ and R² is selected from the group consisting of ═O, —OH, thiol, amino, ester, halogen, nitro or nitrile groups and wherein at least one of substituents R¹ und R² is selected from hydrogen, alkyl or alkylene groups wherein compounds of formula (I) 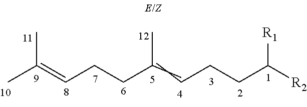
wherein R¹ and R² are as defined above,
are cyclized with an enzyme mutant in accordance with claim 1.

14. Use of an enzyme mutant in accordance with claim 1 for the cyclization of compounds.

15. The use in accordance with claim 14 for the manufacture of dihydroionone derivatives, in particular (+)-γ-dihydroionone, starting from geranylacetone, nerylacetone, calmusal, calmusol or mixtures thereof.
